(19) United States
(12) Patent Application Publication
(10) Pub. No.: US 2024/0382405 A1
(43) Pub. Date: Nov. 21, 2024

(54) POLYESTER ELASTOMERS FROM DI-CARBOXYLIC ACID OR TRI-CARBOXYLIC ACID, MONOCARBOXYLIC ACID, AND POLYOL FOR COSMETIC AND PERSONAL CARE APPLICATIONS

(71) Applicant: MOMENTIVE PERFORMANCE MATERIALS INC., Niskayuna, NY (US)

(72) Inventors: Xu QIN, Northvale, NJ (US); Greg WANG, Vienna, WV (US); Mark David WESTMEYER, Marietta, OH (US); Polina TSUPKO, Bronx, NY (US); Amar PAWAR, Briarcliff Manor, NY (US); Sigfredo GONZALEZ, Danbury, CT (US); Benjamin FALK, Yorktown Heights, NY (US); Alok SARKAR, Bangalore (IN)

(21) Appl. No.: 18/618,683

(22) Filed: Mar. 27, 2024

Related U.S. Application Data
(60) Provisional application No. 63/492,429, filed on Mar. 27, 2023.

(51) Int. Cl. A61K 8/85; A61K 8/04; A61Q 1/02; A61Q 1/06; A61Q 17/04; A61Q 19/00; C08G 63/52; C08G 63/81; C08G 63/87

(52) U.S. Cl. CPC A61K 8/85; A61K 8/042; A61Q 1/02; A61Q 1/06; A61Q 17/04; A61Q 19/00; C08G 63/52; C08G 63/81; C08G 63/87; C08G 2220/00

(57) ABSTRACT

The present disclosure provides polyester elastomers, polyester elastomer compositions, and methods of preparing polyester elastomers and polyester elastomer compositions. The polyester elastomers in the present disclosure are prepared by reacting at least one di-carboxylic acid or tri-carboxylic acid, at least one mono-carboxylic acid, and at least one polyol. The crosslinked polyester elastomers can be formulated into various personal care formulations.

### FIELD

The present disclosure provides polyester elastomers, polyester elastomer compositions, and methods of preparing such polyester elastomers and compositions. These elastomers are prepared by reacting at least one di-carboxylic acid or tri-carboxylic acid, at least one mono-carboxylic acid, and at least one polyol. Further, these polyester elastomers can be converted to polyester elastomer powders and gels. These polyester elastomers, polyester elastomer powders, and polyester elastomer gels can be biodegradable and produced from biorenewable raw materials. Further, these polyester elastomer powders and gels provide advantageous properties when combined with various personal care products.

### BACKGROUND

The personal care industry thrives on being able to deliver multiple performance products based on mixtures of several components, with each having performance characteristics important to or desirable in the final formulation. Silicone gels are commonly added in a variety of personal care formulations to enhance their aesthetics with respect to sensory, texture, rheology, and optical performance. See for example, U.S. Pat. Nos. 4,987,169; 5,654,362; 5,760,116; 6,423,322; and 5,811,487.

Crosslinked polymers are added to manipulate the sensory, texture, rheology, and optical performance of a variety of cosmetic products. Silicone elastomers are particularly important because they can form elastic particles of three-dimensional polymeric dimethicone and provide a beneficial sensory, texture, and optical effect to cosmetic products. However, traditional silicone elastomers have limited versatility in terms of compatibility with polar solvents or emollients such as hydrocarbon oils, plant-based oils, glycerin, and water. Therefore, although the performance of silicone elastomers is unparalleled, there is a demand for alternatives to silicone elastomers. In particular, there is a demand in the marketplace for non-silicone based elastomer materials. US20210059924A1 disclosed a polyurethane elastomeric rubber composition containing a bio-based polyol cross-linked with a bio-based isocyanate. The cross-linked polyurethane elastomer rubber is in further aspect of the invention included in a gel after being milled in the presence of a bio-based emollient or mixture of bio-based emollients. The polyurethane elastomeric gel has good compatibility with cosmetic and natural oils and can be used as a gelling agent for these oils among other desirable cosmetic formulary roles.

Polyesters are a class of compounds that contain an ester functional group in their polymer chain. The ester group can be hydrolyzed when treated with certain biological catalysts or certain mixed cultures of microorganisms which renders a large number of polyesters biodegradable. There is a growing interest in recent years to design and develop biobased polyesters from renewable resources as emollients, emulsifiers, film formers, or other functional ingredients for personal care applications. See for example, U.S. Pat. Nos. 8,414,906; 9,334,358; 6,540,987; and 7,820,758. However, no polyester elastomer or polyester elastomer gel has yet been reported that provides multiple benefits to consumers as a substitute to silicone gel.

### SUMMARY

The present disclosure provides crosslinked polyester elastomers comprising the reaction product of at least one di-carboxylic acid or tri-carboxylic acid, at least one mono-carboxylic acid, and at least one polyol. In an aspect, the polyester elastomers are prepared by an esterification reaction between at least one di-carboxylic acid or tri-carboxylic acid, at least one mono-carboxylic acid, and at least one polyol that creates a crosslinked polymer structure. The polyester elastomers can further be swollen under shear force with a low molecular weight emollient or solvent to form a uniform polyester gel or paste having a wide viscosity range. These elastomers are expected to deliver superior performance benefits such as improved sensory, structuring, and rheological performance compared to their analogous elastomers disclosed previously. In another aspect, this invention relates to a personal care composition containing such high purity polyesters elastomers.

In a first aspect, the present disclosure provides a polyester elastomer comprising the reaction product of
(i) at least one di-carboxylic acid or tri-carboxylic acid;
(ii) at least one mono-carboxylic acid; and
(iii) at least one polyol.

In an aspect, the present disclosure provides an elastomer prepared by reacting:
(i) at least one di-carboxylic acid of formula (IA)

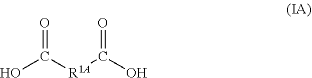

wherein
R^{1A} is C₂-C₅₂ alkyl group, C₂-C₅₂ heteroalkyl group, C₂-C₅₂ alkene group, C₂-C₅₂ heteroalkene group, C₃-C₅₂ cyclic group, or C₂-C₅₂ heterocyclic group; or
at least one tri-carboxylic acid of formula (IB)

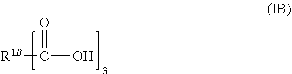

wherein
R^{1B} is C₂-C₅₂ alkyl group, C₂-C₅₂ heteroalkyl group, C₂-C₅₂ alkene group, C₂-C₅₂ heteroalkene group, C₃-C₅₂ cyclic group, or C₂-C₅₂ heterocyclic group;
(ii) at least one mono-carboxylic acid of formula (II)

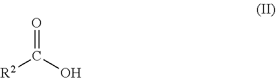

wherein
R² is C₂-C₅₂ alkyl group, C₂-C₅₂ heteroalkyl group, C₂-C₅₂ alkene group, C₂-C₅₂ heteroalkene group, C₃-C₅₂ cyclic group, or C₂-C₅₂ heterocyclic group; and
(iii) at least one polyol of formula (III)

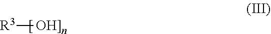

wherein
R³ is C₃-C₅₀ alkyl group, C₃-C₅₀ heteroalkyl group, C₃-C₅₀ alkene group, C₃-C₅₀ heteroalkene group, C₃-C₅₀ cyclic group, or C₃-C₅₀ heterocyclic group; and
n is an integer from 2 to 10.

In an aspect, the present disclosure provides a method of preparing an polyester elastomer comprising reacting:
(i) at least one di-carboxylic acid or tri-carboxylic acid;
(ii) at least one mono-carboxylic acid; and
(iii) at least one polyol.

In an aspect, the preparation of polyester elastomer is without solvent or emollient. In an aspect, the preparation of polyester elastomer is with solvent or emollient as defined herein.

In an aspect, polyester elastomer is only comprised of polyester. In an aspect, polyester elastomer is only comprised of crosslinked polyester. In an aspect, polyester elastomer is comprised of crosslinked polyester and non-crosslinked polyester.

In an aspect, polyester elastomer is comprised of polyester and solvent or emollient. In an aspect, polyester elastomer is comprised of crosslinked polyester and solvent or emollient. In an aspect, polyester elastomer is comprised of crosslinked polyester, non-crosslinked polyester, and solvent or emollient.

In an aspect, the polyester elastomer is a powder.

The polyester elastomers form crosslinked polymer networks. As is well-known to a skilled person in the art such crosslinked polymer are not (completely) soluble and certain methods are available to characterize them, including for example sol-gel analysis (determination of the gel fraction), swelling ratio analysis (determination of the swelling ratio), and mechanical analysis (e.g. determination of the modulus) (see e.g. Polym. Chem., 2024, 15, 219-247).

In an aspect, the fraction of the polyester elastomer which is not soluble in ethyl acetate (gel fraction) is greater than or equal to 20%. In an aspect, the fraction of polyester elastomer which is not soluble in ethyl acetate (gel fraction) is greater than or equal to 40%. In an aspect, the fraction of the polyester elastomer which is not soluble in ethyl acetate (gel fraction) is greater than or equal to 50%. In an aspect, the fraction of the polyester elastomer which is not soluble in ethyl acetate (gel fraction) is greater than or equal to 60%. In an aspect, the fraction of the polyester elastomer which is not soluble in ethyl acetate (gel fraction) is greater than or equal to 70%. The gel fraction is suitably defined as

$$\\ Gel fraction ( \% ) = 100 \times \frac{weight of dried gel ( insoluble residue of the extraction )}{total weight of the polyester elastomer used in the extraction} .$$

In an aspect, the gel fraction can be determined with an extraction method such as the Soxhlet extraction described herein.

In an aspect, polyester elastomer composition is comprised of polyester elastomer and solvent or emollient as defined herein. In an aspect, polyester elastomer composition is comprised of polyester elastomer without solvent or emollient as defined herein. In an aspect, polyester elastomer composition is gel or powder.

In an aspect, the polyester elastomer composition is a polyester elastomer combined with one or more solvents or emollients, which can be converted to a polyester elastomer gel (the swollen polyester elastomer), for example, by applying a shear force to the composition.

In an aspect, the present disclosure provides for the use of a gel, or a powder prepared from a polyester elastomer described herein, in the manufacture of a personal care formulation.

### DETAILED DESCRIPTION

### I. Definitions

Unless otherwise indicated, any atom with unsatisfied valences is assumed to have hydrogen atoms sufficient to satisfy the valences.

It is to be noted that the term “a” or “an” entity refers to one or more of that entity; for example, “a nucleic acid sequence,” is understood to represent one or more nucleic acid sequences, unless stated otherwise. As such, the terms “a” (or “an”), “one or more,” and “at least one” can be used interchangeably herein.

Furthermore, “and/or”, where used herein, is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, the following definitions shall apply unless otherwise indicated. For purposes of the present disclosure, the chemical elements are identified in accordance with the Periodic Table of the Elements, CAS version, and the Handbook of Chemistry and Physics, 75^{th} Ed. 1994. Additionally, general principles of organic chemistry are described in “Organic Chemistry,” Thomas Sorrell, University Science Books, Sausalito: 1999, and “March's Advanced Organic Chemistry,” 6^{th} Ed., Smith, M. B. and March, J., eds. John Wiley & Sons, New York: 2007, the entire contents of which are hereby incorporated by reference in their entireties.

The term “hydrocarbon”, as used herein by itself or as part of a group, refers to a straight- or branched-chain aliphatic series of one to two hundred carbon atoms, i.e., a C₁-C₂₀₀ hydrocarbon, or the number of carbon atoms designated, e.g., a C₁ hydrocarbon such as a methyl, a C₂ hydrocarbon such as ethyl, etc. In one embodiment, the hydrocarbon is a C₂-C₂₀₀ hydrocarbon group. In an embodiment, the hydrocarbon is a C₆-C₆₀ hydrocarbon group. In an embodiment, the hydrocarbon is a C₆-C₆₀ hydrocarbon group. In an embodiment, the hydrocarbon is a C₂-C₆₀ hydrocarbon group. In an embodiment, the hydrocarbon is a C₅-C₂₂ hydrocarbon group. Examples of hydrocarbon groups include butyl, octyl, decyl, lauryl, cetyl (palmityl), and stearyl.

The term “alkyl”, as used herein by itself or as part of a group, refers to a straight- or branched-chain aliphatic hydrocarbon containing one to two hundred carbon atoms, i.e., a C₂-C₂₀₀ alkyl, or the number of carbon atoms designated, e.g., a C₁ alkyl such as methyl, a C₂ alkyl such as ethyl, etc. In one embodiment, the alkyl is a C₂-C₂₀₀ alkyl group. In another embodiment, the alkyl is a C₆-C₆₀ alkyl group. In another embodiment, the alkyl is a C₂-C₆₀ alkyl group. In another embodiment, the alkyl is a C₅-C₂₂ alkyl group. Examples of alkyl groups include butyl, octyl, decyl, lauryl, cetyl (palmityl), and stearyl.

The term “alkene”, as used herein by itself or as part of a group, refers to an alkyl group containing one, two, three, or more carbon-to-carbon double bonds. In one embodiment, the alkene group is a C₂-C₂₀₀ alkylene group. In another embodiment, the alkene group is a C₆-C₆₀ alkene group. In another embodiment, the alkene group is a C₂-C₆₀ alkene group. In another embodiment, the alkene group is a C₅-C₂₂ alkene group.

The term “alkyne”, as used herein by itself or as part of a group, refers to an alkyl group containing one, two, three, or more carbon-to-carbon triple bonds. In another embodiment, the alkyne is a C₂-C₂₀₀ alkyne group.

The term “cyclic”, as used herein by itself or as part of a group, refers to a stable cyclic compound containing three or more atoms. In an embodiment, the cyclic is a C₃-C₂₀₀ cyclic group. In an embodiment, the cyclic is a C₆-C₆₀ cyclic group. In an embodiment, the cyclic is a C₅-C₂₂ cyclic group. Examples of cyclic compounds include benzene, cyclopentane, and cyclohexane.

The term “heteroalkyl”, as used herein by itself or as part of a group, refers to a stable straight or branched chain alkyl radical containing two to two hundred carbon atoms and at least one heteroatom, which can be the same or different, selected from O, N, or S, wherein the sulfur atom(s) can optionally be oxidized. The heteroatoms can be placed at any interior position of the heteroalkyl group or at a position at which the heteroalkyl group is attached to the remainder of the molecule. In an embodiment, the heteroalkyl is a C₆-C₆₀ heteroalkyl group. In an embodiment, the heteroalkyl is a C₂-C₆₀ heteroalkyl group. Examples of heteroalkyl compounds include succinyl, adipoyl, and sebacoyl.

The term “heteroalkene”, as used herein by itself or as part of a group, refers to a stable straight or branched chain alkene radical containing two to two hundred carbon atoms and at least one heteroatom, which can be the same or different, selected from O, N, or S, wherein the sulfur atom(s) can optionally be oxidized. The heteroatom can be placed at any interior position of the heteroalkyl group or at a position at which the heteroalkyl group is attached to the remainder of the molecule. In an embodiment, the heteroalkene is a C₆-C₆₀ heteroalkene group. In an embodiment, the heteroalkene is a C₂-C₆₀ heteroalkene group. Examples of heteroalkene compounds include oleoyl, ricinolyl, and linoleoyl.

The term “heteroalkyne”, as used herein by itself or as part of a group, refers to a stable straight or branched chain alkyne radical containing two to two hundred carbon atoms and at least one heteroatom, which can be the same or different, selected from O, N, or S, wherein the sulfur atom(s) can optionally be oxidized. The heteroatom can be placed at any interior position of the heteroalkyl group or at a position at which the heteroalkyl group is attached to the remainder of the molecule.

The term “heterocyclic”, as used herein by itself or as part of a group, refers to a stable cyclic compound containing two or more carbons atoms and at least one heteroatom, which can be the same or different, selected from O, N, or S, wherein the sulfur atom(s) can optionally be oxidized. In an embodiment, the heterocyclic is a C₂-C₂₀₀ heterocyclic group. In an embodiment, the heterocyclic is a C₆-C₆₀ heterocyclic group. In an embodiment, the heterocyclic is a C₅-C₂₂ heterocyclic group. Examples of heterocyclic compounds include furan, oxolane, and thiophene.

As used herein, the term “olefin” refers to any species having at least one ethylenic double bond such as normal and branched chain aliphatic olefins, cycloaliphatic olefins, aryl substituted olefins, and the like. An olefin can comprise terminal double bond(s) (“terminal olefin”) and/or internal double bond(s) (“internal olefin”) and can be cyclic or acyclic, linear or branched, optionally substituted. The total number of carbon atoms can be from 1 to 100, or from 1 to 40; the double bonds can be unsubstituted or mono-, bi-, tri- or tetrasubstituted.

As used herein, the term “polyolefin” refers to a homopolymer or copolymer of ethylene, propylene, butenes and other unsaturated aliphatic hydrocarbons, vinyl esters (e.g. vinyl acetate), or (meth)acrylics (e.g. butyl acrylate, acrylic acid). Generally, the polyolefin will be a polymer of ethylene, propylene or copolymer thereof, or a copolymer of ethylene or propylene with one or more C₄-C₁₂ α-olefin aliphatic comonomers.

A gel is a disperse system comprising at least two components: a solid component and a liquid component. The solid component forms a sponge-like, three-dimensional network whose pores are filled by a liquid. The liquid component is thus immobilized in the solid. In the gel of the invention the solid component is a three-dimensional network formed of the cross-linked polyester elastomer, and the liquid component is formed of one or more solvents or emollients as defined herein. A gel is a semi-solid that can have properties ranging from soft and weak to hard and tough. Gels are also defined as a substantially dilute cross-linked system.

An elastomer gel is thus made from the elastomer powders or particles swelled or dispersed in a liquid, such as a solvent or an emollient, to form a gel. The ability to swell is commonly expressed by the swelling ratio as explained herein.

Various aspects of the disclosure are described in greater detail below.

### II. Polyester Elastomers

In one aspect, the present disclosure is directed to an elastomer comprising the reaction product of
(i) at least one di-carboxylic acid or tri-carboxylic acid;
(ii) at least one mono-carboxylic acid; and
(iii) at least one polyol.

In an aspect, the elastomer is a polyester elastomer. In an aspect, the elastomer is a crosslinked polyester elastomer.

In an aspect, at least one of the components (i) and/or (iii) has a functionality of >2. In aspect, the polyol (iii) has a functionality of ≥3.

In an aspect, the component (i) has a functionality of 2 (di-carboxylic acid) and the polyol (iii) has a functionality of ≥3.

### DESCRIPTION OF THE FIGURES

FIG. 1 is a bar graph showing the time needed for the polyester elastomer to reach 60% gel fraction measured by Soxhlet extraction in ethyl acetate described herein, prepared with 4 different ratios of monomers A (C₃₆ dimer acid), B (oleic acid), and C (diglycerol) under nitrogen protection at 140° C. No solvent or emollient was used. The molar ratio of B to C was kept constant.

FIG. 2 is a bar graph showing the swelling value (or ratio), measured as described herein, of polyester elastomers in coco-caprylate/caprate prepared with 4 different ratios of monomers A (C₃₆ dimer acid), B (oleic acid), and C (diglycerol) under nitrogen protection at 140° C. No solvent or emollient is used. The molar ratio of B to C was kept constant.

FIG. 3 is a bar graph showing the time needed for crosslinked polyester to reach 60% gel fraction as measured by Soxhlet extraction in ethyl acetate described herein, with 4 different ratios of monomers A (C₃₆ dimer acid), B (oleic acid), and C (diglycerol) under nitrogen protection at 140° C. No solvent or emollient was used. The molar ratio of A to C was kept constant.

FIG. 4 is a bar graph showing the swelling value (or ratio), measured as described herein, of polyester elastomers in coco-caprylate/caprate prepared with 4 different ratios of monomers A (C₃₆ dimer acid), B (oleic acid), and C (diglycerol) under nitrogen protection at 140° C. No solvent or emollient was used.

FIG. 5 is a line graph showing the rheology profile (measured as described herein) of the polyester elastomer gel prepared by processing the polyester elastomer of Example 1 with coco-caprylate/caprate emollient.

FIG. 6 is a line graph showing the particle size distribution (determined as described herein) of the polyester elastomer gel prepared by processing the polyester elastomer of Example 1 with coco-caprylate/caprate solvent or emollient.

### A. COMPONENTS

### 1. Di-carboxylic Acids

In an aspect, the at least one di-carboxylic acid is a compound of formula (IA)

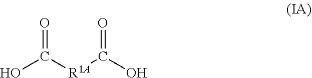

wherein
R^{1A} is C₂-C₅₂ alkyl group, C₂-C₅₂ heteroalkyl group, C₂-C₅₂ alkene group, C₂-C₅₂ heteroalkene group, C₃-C₅₂ cyclic group, or C₂-C₅₂ heterocyclic group.

In an aspect, the di-carboxylic acid is a compound of formula (IA), wherein R^{1A} is C₄-C₃₄ alkyl group, C₄-C₃₄ heteroalkyl group, C₄-C₃₄ alkene group, C₄-C₃₄ heteroalkene group, C₄-C₃₄ cyclic group, or C₄-C₃₄ heterocyclic group.

In an aspect, the di-carboxylic acid is a compound of formula (IA), wherein R^{1A} is C₄-C₃₄ alkyl group.

In an aspect, the di-carboxylic acid is selected from the group consisting of succinic acid, glutaric acid, adipic acid, pimelic acid, suberic acid, azelaic acid, sebacic acid, brassylic acid, dodecanedioic acid, C₂₁ dimer acid, C₃₆ dimer acid, maleic acid, fumaric acid, traumatic acid, and combinations thereof. In an aspect, the di-carboxylic acid is dilinoleic acid. In an aspect, the di-carboxylic acid is a C₃₆ dimer acid. In an aspect, the di-carboxylic acid is a hydrogenated C₃₆ dimer acid.

In an aspect, the di-carboxylic acid is biobased or naturally derived.

### 2. Tri-carboxylic Acids

In an aspect, the at least one tri-carboxylic acid is a compound of formula (IB)

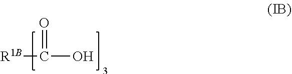

wherein
R^{1B} is C₂-C₅₂ alkyl group, C₂-C₅₂ heteroalkyl group, C₂-C₅₂ alkene group, C₂-C₅₂ heteroalkene group, C₃-C₅₂ cyclic group, or C₂-C₅₂ heterocyclic group.

In an aspect, the tri-carboxylic acid is a compound of formula (IB), wherein R^{1B} is C₄-C₃₄ alkyl group, C₄-C₃₄ heteroalkyl group, C₄-C₃₄ alkene group, C₄-C₃₄ heteroalkene group, C₄-C₃₄ cyclic group, or C₄-C₃₄ heterocyclic group.

In an aspect, the tri-carboxylic acid is a compound of formula (IB), wherein R^{1B} is C₄-C₃₄ alkyl group.

In an aspect, the tri-carboxylic acid is selected from the group consisting of citric acid, C₅₄ trimer acid, and hydrogenated C₅₄ trimer acid. In an aspect, the tri-carboxylic acid is a C₅₄ trimer acid.

In an aspect, the di-carboxylic acid is biobased or naturally derived.

### 3. Mono-carboxylic Acids

In an aspect, the at least one mono-carboxylic acid is a compound of formula (II)

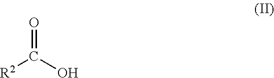

wherein
R² is C₂-C₅₂ alkyl group, C₂-C₅₂ heteroalkyl group, C₂-C₅₂ alkene group, C₂-C₅₂ heteroalkene group, C₃-C₅₂ cyclic group, or C₂-C₅₂ heterocyclic group.

In an aspect, the mono-carboxylic acid is a compound is formula (II), wherein R² is C₅-C₂₁ alkyl group, C₅-C₂₁ heteroalkyl group, C₅-C₂₁ alkene group, C₅-C₂₁ heteroalkene group, C₅-C₂₁ cyclic group, or C₅-C₂₁ heterocyclic group.

In an aspect, the mono-carboxylic acid is selected from the group consisting of caprylic acid, capric acid, lauric acid, myristic acid, palmitic acid, stearic acid, isostearic acid, behenic acid, oleic acid, linoleic acid, linolenic acid, and combinations thereof. In an aspect, the mono-carboxylic acid is oleic acid. In an aspect, the mono-carboxylic acid is isostearic acid.

In an aspect, the mono-carboxylic acid is biobased or naturally derived.

### 4. Polyols

In an aspect, the at least one polyol is a compound of formula (III)

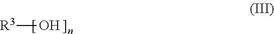

wherein
R³ is C₃-C₅₀ alkyl group, C₃-C₅₀ heteroalkyl group, C₃-C₅₀ alkene group, C₃-C₅₀ heteroalkene group, C₃-C₅₀ cyclic group, or C₃-C₅₀ heterocyclic group; and

n is an integer from 2 to 10.

In an aspect, the polyol is a compound of formula (III), wherein R³ is C₃-C₅₀ alkyl group, C₃-C₅₀ heteroalkyl group, C₃-C₅₀ alkene group, or C₃-C₅₀ heteroalkene group; and n is an integer from 2 to 10.

In an aspect, the polyol is a compound of formula (III), wherein R³ is C₃-C₂₀ alkyl group, C₃-C₂₀ heteroalkyl group, C₃-C₂₀ alkene group, or C₃-C₂₀ heteroalkene group; and n is an integer from 2 to 10.

In an aspect, the polyol is a compound of formula (III), wherein n is an integer from 2 to 6. In an aspect, the polyol is a compound of formula (III), wherein n is 2, 3, 4, 5, or 6. In an aspect, the polyol is a compound of formula (III), wherein n is an integer from 3 to 10. In an aspect, the polyol is a compound of formula (III), wherein n is an integer from 3 to 6.

In an aspect, the polyol is selected from the group consisting of glycerol, diglycerol, polyglycerol, polyglycerol-3, sorbitol, castor oil, hydrogenated castor oil, sugar alcohol, monosaccharide, disaccharides, oligosaccharide, polysaccharides, tannin, gallic acid, 1,2-propanediol, 1,3-propanediol, 1,4-butanediol, 1,2-pentanediol, 1,3-pentanediol, 1,4-pentanediol, 1,5-pentanediol, 1,2-hexanediol, 1,5-hexanediol, 1,6-hexanediol, C₃₆ dimer diol, hydrogenated C₃₆ dimer diol, and combinations thereof. In an aspect, the polyol is hydrogenated castor oil. In an aspect, the polyol is diglycerol. In an aspect, the polyol is polyglycerol-3. In an aspect, the polyol is polyglycerol-4.

In an aspect, the polyol is biobased or naturally derived.

### B. RATIO OF COMPONENTS

In an aspect, the elastomer comprises a defined molar ratio of carboxyl functional groups (—COOH) from mono-carboxylic acid to hydroxyl functional groups (—OH) from polyol. It has been found that the molar ratio of carboxyl functional groups (—COOH) from mono-carboxylic acid to hydroxyl functional groups (—OH) from the polyol influences the performance of the polyester elastomer and the performance of the gel made from the polyester elastomer.

In an aspect, the molar ratio of carboxyl functional groups (—COOH) from mono-carboxylic acid to hydroxyl functional groups (—OH) from polyol is from about 1:2 to about 1:16. In an aspect, the molar ratio of carboxyl functional groups (—COOH) from mono-carboxylic acid to hydroxyl functional groups (—OH) from polyol is from about 1:2 to about 1:14. In an aspect, the molar ratio of carboxyl functional groups (—COOH) from mono-carboxylic acid to hydroxyl functional groups (—OH) from polyol is from about 1:2 to about 1:10. In an aspect, the molar ratio of carboxyl functional groups (—COOH) from mono-carboxylic acid to hydroxyl functional groups (—OH) from polyol is from about 1:2 to about 1:8. In an aspect, the molar ratio of carboxyl functional groups (—COOH) from mono-carboxylic acid to hydroxyl functional groups (—OH) from polyol is from about 1:2 to about 1:5. In an aspect, the molar ratio of carboxyl functional groups (—COOH) from mono-carboxylic acid to hydroxyl functional groups (—OH) from polyol is about 1:16, about 1:15, about 1:14, about 1:13, about 1:12, about 1:11, about 1:10, about 1:9, about 1:8, about 1:7, about 1:6, about 1:5, about 1:4, about 1:3, or about 1:2.

In an aspect, the molar ratio of carboxyl functional groups (—COOH) from di-carboxylic acid or tri-carboxylic acid to hydroxyl functional groups (—OH) from polyol is from about 1.5:1 to about 1:4. In an aspect, the ratio of carboxyl functional groups (—COOH) from di-carboxylic acid or tri-carboxylic acid to hydroxyl functional groups (—OH) from polyol is from about 1.5:1 to about 1:2. In an aspect, the ratio of carboxyl functional groups (—COOH) from di-carboxylic acid or tri-carboxylic acid to hydroxyl functional groups (—OH) from polyol is from about 1.5:1 to about 1:1.5. In an aspect, the ratio of carboxyl functional groups (—COOH) from di-carboxylic acid or tri-carboxylic acid to hydroxyl functional groups (—OH) from polyol is from about 1.5:1 to about 1:1.25. In an aspect, the ratio of carboxyl functional groups (—COOH) from di-carboxylic acid or tri-carboxylic acid to hydroxyl functional groups (—OH) from polyol is about 1.5:4, about 1.5:3, about 1.5:2, about 1:1, about 1:2, about 1:3, or about 1:4.

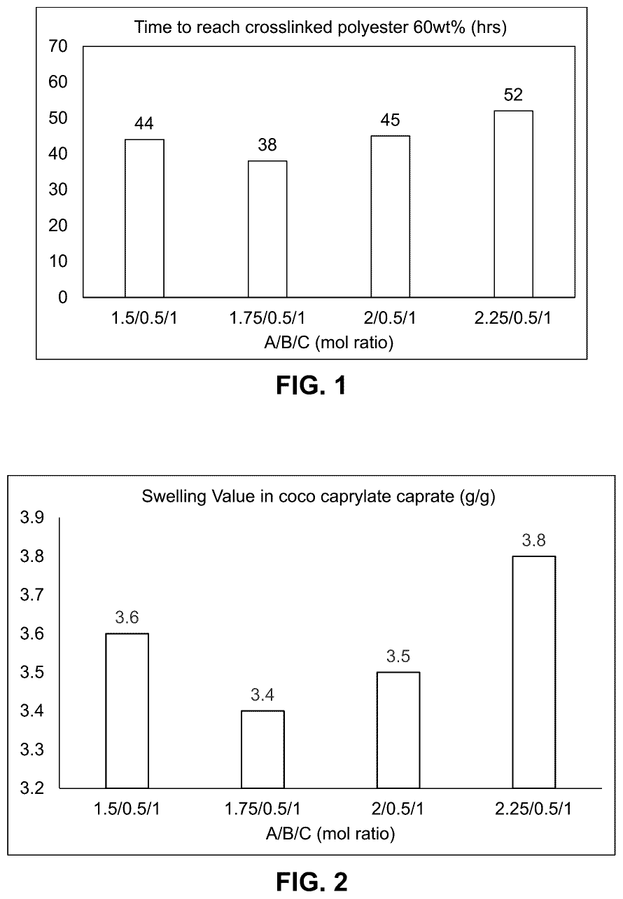

The results in FIGS. 1, 2, 3 and 4 show that if the mole ratio of di-carboxylic acid or tri-carboxylic acid to polyol is kept constant, the amount of mono-carboxylic acid has substantial impact on the weight percentage of crosslinked polyester in resultant polyester elastomer, and the swelling value (i.e., the swelling ratio) of the polyester elastomer in coco-caprylate/caprate. The swelling value of the polyester elastomer is an important indication of how the polyester elastomer performs in personal care formulations.

In an aspect, the conversion of carboxylic acid functional groups (—COOH) to ester functional groups (—CO(O)—) is no less than 80% by mole. The percent conversion is calculated by titration of carboxylic acid functional groups (—COOH) with 0.1 N KOH in isopropanol.

### II. Methods of Preparing the Polyester Elastomer

### 1. Esterification Reaction

In one aspect, the present disclosure is directed to a method of preparing an elastomer comprising reacting:
(i) at least one di-carboxylic acid or tri-carboxylic acid;
(ii) at least one mono-carboxylic acid; and
(iii) at least one polyol.

In an aspect, the elastomer prepared is a polyester elastomer. In an aspect, the elastomer prepared is a crosslinked polyester elastomer.

In an aspect, the preparation of the elastomer is under nitrogen protection, is under vacuum, or is a combination thereof.

In an aspect, the elastomer is prepared by reacting:
(i) at least one di-carboxylic acid or tri-carboxylic acid, optionally wherein the di-carboxylic acid or tri-carboxylic acid is an activated di-carboxylic or activated tri-carboxylic acid;
(ii) at least one mono-carboxylic acid; and
(iii) at least one polyol.

In an aspect, the elastomer is prepared by reacting:
(i) at least one activated di-carboxylic acid or tri-carboxylic acid;
(ii) at least one mono-carboxylic acid; and
(iii) at least one polyol.

In an aspect, wherein the reaction comprised an activated di-carboxylic acid or tri-carboxylic acid, the preparation of the elastomer further comprises addition of water to quench the activating agent from the reaction.

In an aspect, esterification is conducted in solvent or emollient. In another aspect, esterification is conducted in more than one solvent or emollient.

In an aspect, esterification is carried out in the absence of a solvent or emollient.

### 2. Ratio of Components

In an aspect, the preparation of the elastomer comprises a defined ratio of di-carboxylic acid or tri-carboxylic acid (A) to polyol (C), and a defined ratio of mono-carboxylic acid (B) to polyol (C). It has been found that the ratio of A/C and B/C influences the performance of the polyester elastomer and the performance of the gel made from the polyester elastomer.

In an aspect, the molar ratio of carboxyl functional groups (—COOH) from mono-carboxylic acid (B) to hydroxyl functional groups (—OH) from polyol (C) is from about 1:2 to about 1:16. In an aspect, the molar ratio of carboxyl functional groups (—COOH) from mono-carboxylic acid (B) to hydroxyl functional groups (—OH) from polyol (C) is from about 1:2 to about 1:14. In an aspect, the molar ratio of carboxyl functional groups (—COOH) from mono-carboxylic acid (B) to hydroxyl functional groups (—OH) from polyol (C) is from about 1:2 to about 1:10. In an aspect, the molar ratio of carboxyl functional groups (—COOH) from mono-carboxylic acid (B) to hydroxyl functional groups (—OH) from polyol (C) is from about 1:2 to about 1:8. In an aspect, the molar ratio of carboxyl functional groups (—COOH) from mono-carboxylic acid (B) to hydroxyl functional groups (—OH) from polyol (C) is from about 1:2 to about 1:5. In an aspect, the molar ratio of carboxyl functional groups (—COOH) from mono-carboxylic acid (B) to hydroxyl functional groups (—OH) from polyol (C) is about 1:16, about 1:15, about 1:14, about 1:13, about 1:12, about 1:11, about 1:10, about 1:9, about 1:8, about 1:7, about 1:6, about 1:5, about 1:4, about 1:3, or about 1:2.

In an aspect, the molar ratio of carboxyl functional groups (—COOH) from di-carboxylic acid or tri-carboxylic acid (A) to hydroxyl functional groups (—OH) from polyol (C) is from about 1.5:1 to about 1:4. In an aspect, the ratio of carboxyl functional groups (—COOH) from di-carboxylic acid or tri-carboxylic acid (A) to hydroxyl functional groups (—OH) from polyol (C) is from about 1.5:1 to about 1:2. In an aspect, the ratio of carboxyl functional groups (—COOH) from di-carboxylic acid or tri-carboxylic acid (A) to hydroxyl functional groups (—OH) from polyol (C) is from about 1.5:1 to about 1:1.5. In an aspect, the ratio of carboxyl functional groups (—COOH) from di-carboxylic acid or tri-carboxylic acid (A) to hydroxyl functional groups (—OH) from polyol (C) is from about 1.5:1 to about 1:1.25. In an aspect, the ratio of carboxyl functional groups (—COOH) from di-carboxylic acid or tri-carboxylic acid (A) to hydroxyl functional groups (—OH) from polyol (C) is about 1.5:4, about 1.5:3, about 1.5:2, about 1:1, about 1:2, about 1:3, or about 1:4.

In an aspect, the conversion of carboxyl functional groups (—COOH) to ester functional groups (—CO(O)—) is no less than 80% by mole. The percent conversion is calculated by titration of carboxylic acid functional groups (—COOH) with 0.1N KOH in isopropanol.

In an aspect, esterification is carried out in the absence of a solvent or emollient.

In an aspect, esterification is conducted in solvent or emollient. In another aspect, esterification is conducted in more than one solvent or emollient.

In an aspect, the percentage of solvent or emollient in total raw materials of the esterification reaction is in the range of 0% to 80% by weight. In an aspect, the percentage of solvent or emollient in total raw materials of the esterification reaction is in the range of 0% to 60% by weight. In an aspect, the percentage of solvent or emollient in total raw materials of the esterification reaction is in the range of 0% to 50% by weight. In an aspect, the percentage of solvent or emollient in total raw materials of the esterification reaction is in the range of 0% to 40% by weight. In an aspect, the percentage of solvent or emollient in total raw materials of the esterification reaction is in the range of 0% to 30% by weight. In an aspect, the percentage of solvent or emollient in total raw materials of the esterification is in the range of 0% to 20% by weight. In an aspect, the percentage of solvent or emollient in total raw materials of the esterification reaction is in the range of 0% to 10% by weight. In an aspect, the percentage of solvent or emollient in total raw materials of the esterification reaction is in the range of 10% to 20% by weight. In an aspect, the percentage of solvent or emollient in total raw materials of the esterification reaction is in the range of 20% to 30% by weight.

FIG. 1 is a bar graph showing the gel time of elastomers prepared with 4 different ratios of di-carboxylic acid or tri-carboxylic acid (A), mono-carboxylic acid (B), and polyol (C). The molar ratio of mono-carboxylic acid (B) to polyol (C) was kept constant under nitrogen protection at 140° C. The gel time is the amount of time it takes for the extent of crosslinking of the polyester (gel fraction) to reach 60% by weight of the polyester elastomer as measured by Soxhlet extraction described herein. As shown in FIG. 1, the molar ratio of di-carboxylic acid or tri-carboxylic acid (A) to polyol (C) has a profound impact on gel time in the synthesis of a polyester elastomer. The shortest gel time occurred with a molar ratio of A/B/C between 1.5/0.5/1 and 2/0.5/1.

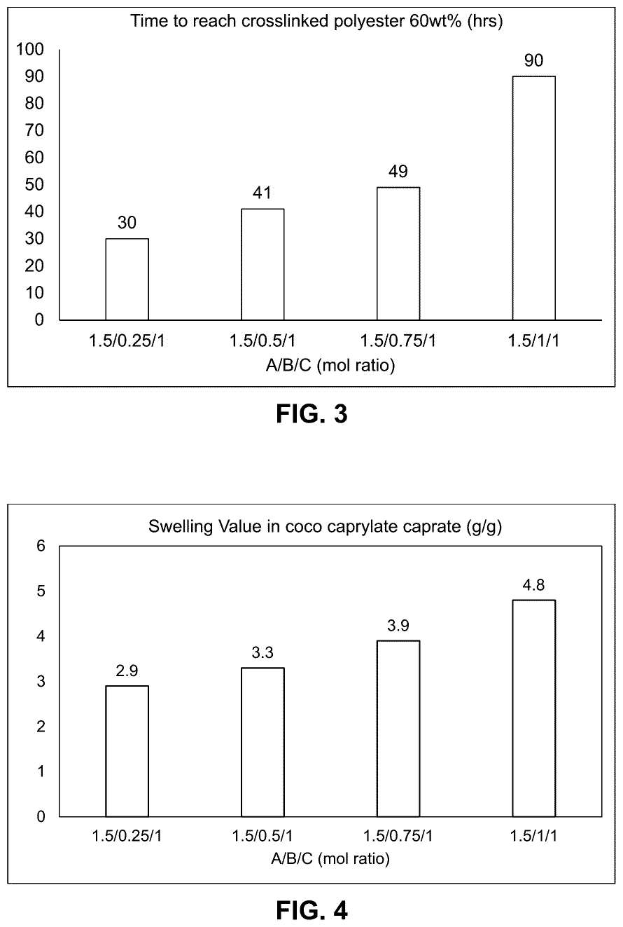

FIG. 3 is a bar graph showing the gel time of elastomers prepared with 4 different ratios of di-carboxylic acid or tri-carboxylic acid (A), mono-carboxylic acid (B), and polyol (C). The molar ratio of di-carboxylic acid or tri-carboxylic acid (A) to polyol (C) was kept constant under nitrogen protection at 140° C. The gel time is the amount of time it takes for the extent of crosslinking of the polyester (gel fraction) to reach 60% by weight of the polyester elastomer measured by Soxhlet extraction described herein. As shown in FIG. 3, the ratio of mono-carboxylic acid (B) to polyol (C) has profound impact on gel time in the synthesis of the polyester elastomer. The shortest gel time occurred with a molar ratio of A/B/C of 1.5/0.25/1.

### 3. Activating Agent

In an aspect, the preparation of the elastomer further comprises an activating agent. In an aspect, the preparation of the elastomer does not comprise an activating agent.

In an aspect, the activating agent is selected from the group consisting of dimethyl dicarbonate, diethyl dicarbonate, dipropyl dicarbonate, di-tertiary-butyl dicarbonate, and combinations thereof.

### 4. Catalyst

In an aspect, the preparation of the elastomer further comprises a catalyst. In an aspect, the preparation of the elastomer does not comprise a catalyst. However, it has been found that when no catalyst is used the reaction times are protracted.

In an aspect, the catalyst is selected from the group consisting of methanesulfonic acid, p-toluenesulfonic acid, benzene sulfonic acid, sulfuric acid, amidosulfonic acid, sulfamic acid, sodium bisulfate, phosphoric acid, hydrochloric acid, hydrobromic acid, nitric acid, sodium hydroxide, potassium hydroxide, sodium methoxide, sodium ethoxide, potassium methoxide, potassium ethoxide, bismuth(III) neodecanoate, bismuth(III) citrate, bismuth(III) chloride, bismuth(III) acetate, bismuth(III) phosphate, tin chloride, tin-pyrone, dibutyltin dilaurate, di-n butyl-oxo-stannane, butyl stannoic acid, zinc chloride, zinc bromide, zinc carboxylic salt, zinc oxide, zinc hydroxy nitrate salt, zinc hydroxy acetate, triethylamine, tripropylamine, cocamidopropyl dimethylamine, stearamidopropyl dimethylamine, isostearamidopropyl dimethylamine, and combinations thereof. In an aspect, the catalyst is p-toluenesulfonic acid, methanesulfonic acid, phosphoric acid, bismuth neodecanoate, or a combination thereof. In an aspect, the catalyst is methanesulfonic acid.

In an aspect, the catalyst is a salt. In an aspect, the catalyst is a salt selected from the group consisting of Yb(OTf)₃, Sc(OTf)₃, Hf(OTf)₄, Bi(OTf)₃, Al(OTf)₃, Zn(OTf)₂, Mg(ClO₄)₂, Cu(OTf)₂, Ti(OCH(CH₃)₂)₄, and combinations thereof.

### 5. Emollient or Solvent

In an aspect, the preparation of the polyester elastomer can occur in the presence of a solvent. The solvent can also act as an emollient, preferably a cosmetic emollient. When the solvent acts also as an emollient it also provides a softening, protecting, moisturizing, and/or lubricating effect to the skin. In an aspect, the solvent or emollient is a biobased or naturally derived. In an aspect, the solvent or emollient is a triglyceride solvent, a mono-ester solvent, a di-ester solvent, a citrate ester solvent, an ether solvent, a carbonate solvent, a hydrocarbon solvent, a silicone solvent, or a combination thereof.

In an aspect, the solvent is a triglyceride solvent of formula (IV)

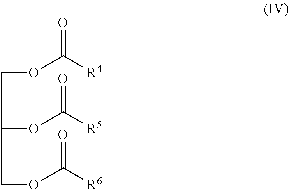

wherein
each R⁴, R⁵, and R⁶ are independently C₁-C₃₅ alkyl group, C₁-C₃₅ heteroalkyl group, C₂-C₃₅ alkene group, or C₂-C₃₅ heteroalkene group.

In an aspect, the solvent is of formula (IV), wherein R⁴, R⁵, and R⁶ are independently C₂-C₁₇ alkyl group or C₂-C₁₇ alkylene group.

In an aspect, the solvent is a triglyceride solvent selected from the group consisting of caprylic/capric triglyceride, triheptanoin, corn oil, soybean oil, olive oil, rape seed oil, cotton seed oil, coconut oil, almond oil, argon oil, rosehip oil, black seed oil, grape seed oil, avocado oil, apricot kernel oil, geranium oil, lavender oil, rosehip oil, macadamia oil, eucalyptus oil, sardine oil, herring oil, safflower oil, linseed oil, sunflower oil, olive oil, canola oil, sesame oil, cottonseed oil, palm oil, rapeseed oil, tung oil, fish oil, peanut oil, cuphea oil, milkweed oil, salicornia oil, whale oil, castor oil, and combinations thereof. In an aspect, the triglyceride solvent is selected from the group consisting of caprylic/capric triglyceride, triheptanoin, and combinations thereof.

In an aspect, the solvent is a mono-ester solvent of formula (V)

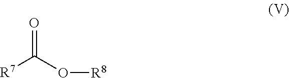

wherein
R⁷ is C₁-C₃₅ alkyl group, C₁-C₃₅ heteroalkyl group, C₂-C₃₅ alkene group, or C₂-C₃₅ heteroalkene group; and
R⁸ is C₁-C₃₅ alkyl group, C₁-C₃₅ heteroalkyl group, C₂-C₃₅ alkene group, or C₂-C₃₅ heteroalkene group.

In an aspect, the solvent is a mono-ester solvent of formula (V), wherein R⁷ is C₅-C₁₇ alkyl group or C₅-C₁₇ alkene group and R⁸ is C₂-C₁₇ alkyl group or C₂-C₁₇ alkene group.

In an aspect, the solvent is a mono-ester solvent selected from the group consisting of coco-caprylate, coco-caprate, jojoba oil, jojoba esters, isopropyl jojobate, ethyl macadamiate, isoamyl laurate, heptyl undecylenate, methylheptyl isostearate, isostearyl isostearate, glyceryl ricinoleate, isostearyl palmitate, myristyl myristate, octyldodecyl myristate, octyldodecyl hydroxystearate, butyl myristate, ethylhexyl cocoate, ethylhexyl palmitate, ethylhexyl stearate, butyl stearate, decyl oleate, isocetyl behenate, isocetyl myristate, isocetyl palmitate, isocetyl stearate, isodecyl oleate, isopropyl isostearate, isopropyl myristate, isopropyl palmitate, oleyl oleate, propylene glycol laurate, octyldodecyl erucate, C₁₂-C₁₃ alkyl lactate, C₁₂-C₁₅ alkyl lactate, isostearyl lactate, glycereth-5-lactate, lauryl lactate, myristyl lactate, oleyl lactate, laureth-2-benzoate, C₁₂-C₁₅ alkyl benzoate, C₁₂-C₁₅ pareth-3-benzoate, dipropylene glycol benzoate, isodecyl salicylate, C₁₂-C₁₅ alkyl salicylate, tridecyl salicylate, ethylhexyl isononanoate, cetyl ethylhexanoate, isononyl isononanoate, isodecyl ethylhexanoate, isodecyl isononanoate, tridecyl ethylhexanoate, isotridecyl isononanoate, isostearyl isononanoate, cetearyl isononanoate, laureth-2-ethylhexanoate, cetearyl ethylhexanoate, isodecyl neopentanoate, isostearyl neopentanoate, nyristyl neopentanoate, isostearyl behenate, octyldodecyl neopentanoate, tridecyl neopentanoate, and combinations thereof. In an aspect, the mono-ester solvent is selected from the group consisting of coco-caprylate/caprate, coco-caprylate, jojoba oil, isoamyl laurate, methylheptyl isostearate, C₁₂-C₁₃ alkyl lactate, C₁₂-C₁₅ alkyl lactate, lauryl lactate, ethylhexyl isononanoate, cetyl ethylhexanoate, isononyl isononanoate, isodecyl ethylhexanoate, isodecyl isononanoate, tridecyl ethylhexanoate, isotridecyl isononanoate, isostearyl isononanoate, cetearyl isononanoate, and combinations thereof. In an aspect, the mono-ester solvent is selected from the group consisting of coco-caprylate/caprate, coco-caprylate, isoamyl laurate, isononyl isononanoate, heptyl undecylenate, jojoba oil, jojoba esters, and combinations thereof.

In an aspect, the solvent is:
(a) a di-ester solvent of formula (VI)

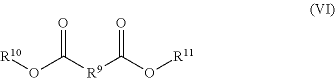

wherein
R⁹ is C₁-C₃₅ alkyl group, C₁-C₃₅ heteroalkyl group, C₂-C₃₅ alkene group, or C₂-C₃₅ heteroalkene group; and
R¹⁰ and R¹¹ are independently C₁-C₃₅ alkyl group, C₁-C₃₅ heteroalkyl group, C₂-C₃₅ alkene group, or C₂-C₃₅ heteroalkene group; or
(b) a di-ester solvent of formula (VII)

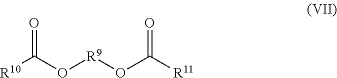

wherein
R⁹ is C₁-C₃₅ alkyl group, C₁-C₃₅ heteroalkyl group, C₂-C₃₅ alkene group, or C₂-C₃₅ heteroalkene group; and
R¹⁰ and R¹¹ are independently H, C₁-C₃₅ alkyl group, C₁-C₃₅ heteroalkyl group, C₂-C₃₅ alkene group, or C₂-C₃₅ heteroalkene group; or
(c) a di-ester solvent of formula (VIII)

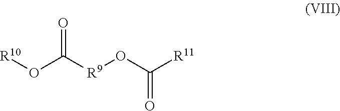

wherein
R⁹ and R¹⁰ are independently C₁-C₃₅ alkyl group, C₁-C₃₅ heteroalkyl group, C₂-C₃₅ alkene group, or C₂-C₃₅ heteroalkene group; and
R¹¹ is H, C₁-C₃₅ alkyl group, C₁-C₃₅ heteroalkyl group, C₂-C₃₅ alkene group, or C₂-C₃₅ heteroalkene group.

In an aspect, the solvent is a di-ester solvent of formula (VI), formula (VII), or formula (VIII), wherein R¹¹ is C₂-C₁₀ alkyl group or C₂-C₁₀ alkene group and R⁹ and R¹⁰ are independently C₁-C₁₂ alkyl group or C₂-C₁₂ alkene group.

In an aspect, the di-ester solvent is selected from the group consisting of diethyl succinate, dibutyl succinate, diethyhexyl succinate, diisopropyl sebacate, dimethyl sebacate, diethyl sebacate, dibutyl sebacate, diisostearyl dimer, diisostearyl malate, isostearyl stearoyl stearate, isocetyl stearoyl stearate, octyldodecyl stearoyl stearate, diethylhexyl malate, diethylhexyl maleate, dipropylene glycol dibenzoate, dicapryl adipate, dicaprylyl maleate, diisopropyl dimer, diisopropyl adipate, diisobutyl adipate, diisopropyl sebacate, diisostearyl dimer, diethyhexyl succinate, diethylene glycol diethylhexanoate, neopentyl glycol dicaprate, propylene glycol dicaprylate/dicaprate, neopentyl glycol diisostearate, neopentyl glycol diethylhexanoate, neopentyl glycol diheptanoate, and combinations thereof. In an aspect, the di-ester solvent is selected from the group consisting of dicapryl adipate, dicaprylyl maleate, diisopropyl adipate, diisobutyl adipate, diethyl succinate, dibutyl succinate, diethyhexyl succinate, diisopropyl sebacate, dimethyl sebacate, diethyl sebacate, dibutyl sebacate, neopentyl glycol diethylhexanoate, neopentyl glycol diheptanoate, and combinations thereof.

In an aspect, the solvent is a citrate ester solvent of formula (IX)

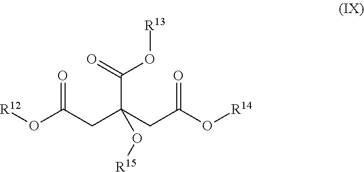

wherein
R¹², R¹³, R¹⁴, and R¹⁵ are independently H, C₁-C₃₅ alkyl group, C₁-C₃₅ heteroalkyl group, C₂-C₃₅ alkene group, or C₂-C₃₅ heteroalkene group, wherein at least one of R¹², R¹³, R¹⁴, and R¹⁵ is not H.

In an aspect, the solvent is a citrate ester solvent of formula (IX), wherein R¹², R¹³, and R¹⁴ are independently C₁-C₁₀ alkyl group or C₂-C₁₀ alkene group and R¹⁵ is an acetyl group.

In an aspect, the solvent is a citrate ester solvent selected from the group consisting of tricaprylyl citrate, triisostearyl citrate, triisocetyl citrate, trioctyldodecyl citrate, triethyl citrate, tributyl citrate, acetyl triethyl citrate, acetyl tributyl citrate, trioctyldodecyl citrate, triisocetyl citrate, and combinations thereof.

In an aspect, the solvent is an ether solvent of formula (X)

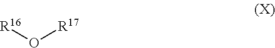

wherein
R¹⁶ and R¹⁷ are independently H, C₂-C₂₀ alkyl group, C₂-C₂₀ heteroalkyl group, C₂-C₂₀ alkene group, or C₂-C₂₀ heteroalkene group, wherein at least one of R¹⁶ and R¹⁷ is not H.

In an aspect, the solvent is an ether solvent of formula (X), wherein R¹⁶ and R¹⁷ are independently C₂-C₂₀ alkyl group.

In an aspect, the solvent is an ether solvent selected from the group consisting of dicaprylyl ether, didecyl ether, panthenyl ethyl ether, dicetyl ether, dimyristyl ether, distearyl ether, dilauryl ether, and combinations thereof. In an aspect, the ether solvent is selected from the group consisting of dicaprylyl ether, didecyl ether, and combinations thereof.

In an aspect, the solvent is a carbonate solvent of formula (XI)

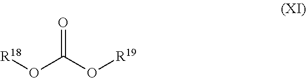

wherein
R¹⁸, and R¹⁹ are independently H, C₂-C₂₀ alkyl group, C₂-C₂₀ heteroalkyl group, C₂-C₂₀ alkene group, or C₂-C₂₀ heteroalkene group.

In an aspect, the solvent is a carbonate solvent of formula (XI), wherein R¹⁸ and R¹⁹ are independently C₂-C₂₀ alkyl group.

In an aspect, the solvent is a carbonate solvent selected from the group consisting of dicaprylyl carbonate, diethyl hexyl carbonate, and combinations thereof.

In an aspect, the solvent is a hydrocarbon with number of carbon atoms from C₄ to C₆₀. In an aspect, the solvent is a hydrocarbon with a number of carbon atoms from C₁₀ to C₅₀. In an aspect, the solvent is a hydrocarbon with a number of carbon atoms from C₂₀ to C₄₀

In an aspect, the solvent is a hydrocarbon solvent selected from the group consisting of farnesene, hydrogenated farnesene, coconut alkanes, coconut/palm kernel alkanes, C₉-C₁₂ alkane, C₁₀-C₁₃ alkane, C₁₂-C₁₇ alkane, C₁₃-C₁₄ alkane, C₁₃-C₁₅ alkane, C₁₄-C₁₇ alkane, C₁₄-C₁₉ alkane, C₁₄-C₂₀ alkane, C₁₄-C₂₂ alkane, C₁₅-C₁₉ alkane, C₂₁-C₂₈ alkane, C₁₇-C₂₃ alkane, C₉-C₁₂ isoalkane, C₉-C₁₃ isoalkane, C₉-C₁₄ isoalkane, C₉-C₁₆ isoalkane, C₁₀-C₁₁ isoalkane, C₁₀-C₁₂ isoalkane, C₁₀-C₁₃ isoalkane, C₁₁-C₁₂ isoalkane, C₁₁-C₁₃ isoalkane, C₁₁-C₁₄ isoalkane, C₁₂-C₁₄ isoalkane, C₁₂-C₁₅ isoalkane, C₁₂-C₂₀ isoalkane, C₁₃-C₁₄ isoalkane, C₁₃-C₁₆ isoalkane, C₁₄-C₁₆ isoalkane, C₁₅-C₁₉ isoalkane, C₁₀-C₁₆ olefin, C₁₂-C₁₈ olefin, C₁₈-C₂₆ olefin, C₂₀ olefin, C₂₀-C₂₄ olefin, C₂₄-C₃₀ olefin, C₂₆-C₂₈ olefin, C₂₆-C₅₄ olefin, C₂₈-C₃₆ olefin, C₂₈-C₅₂ olefin, C₃₀-C₃₈ olefin, C₃₀-C₄₅ olefin, C₄-C₁₂ olefin, C₄. C₆ olefin, C₅-C₆ olefin, hydrogenated poly(C₆/C₁₀/C₁₄ olefin), hydrogenated poly(C₆-C₁₂ olefin), hydrogenated poly(C₆-C₁₄ olefin), hydrogenated poly(C₆-C₂₀ olefin), hydrogenated poly(C₈/C₁₂ olefin), poly(C₂₀-C₂₈ olefin), poly(C₃₀-C₄₅ olefin), poly(C₄-C₁₂ olefin), poly(C₆-C₁₄ olefin), hexadecene, C₃₂ alkane, C₃₂ isoalkane, C₅₄ alkane, C₅₄ isoalkane, diethylhexylcyclohexane, undecane, tridecane, tetradecane, pentadecane, hexadecane, octadecane, docosane, squalane, hydrogenated polyisobutene, polybutene, hydrogenated polydecene, hydrogenated didecene, mineral oil, liquidum, petrolatum, dodecane, isohexadecane, isododecane, isoeicosane, and combinations thereof. In an aspect, the hydrocarbon solvent is selected from the group consisting of squalane, farnesene, hydrogenated farnesene, coconut alkanes, C₉-C₁₂ alkane, C₁₃-C₁₅ alkane, C₁₄-C₁₉ alkane, C₁₄-C₂₀ alkane, C₁₄-C₂₂ alkane, C₁₅-C₁₉ alkane, C₁₃-C₁₆ isoalkane, dodecane, undecane, tridecane, tetradecane, pentadecane, hexadecane, hexadecene, octadecane, squalane, isododecane, isohexadecane, C₃₂ alkane, C₃₂ isoalkane, C₅₄ alkane, C₅₄ isoalkane, and combinations thereof. In an aspect, the hydrocarbon solvent is selected from the group consisting of squalane, hydrogenated farnesene, coconut alkanes, C₉-C₁₂ alkane, C₁₃-C₁₅ alkane, C₁₃-C₁₆ isoalkane, C₁₄-C₁₉ alkane, dodecane, tetradecane, isododecane, hexadecane, octadecane, hexadecene, C₃₂ alkane, C₃₂ isoalkane, C₅₄ alkane, C₅₄ isoalkane, and combinations thereof.

In an aspect, the hydrocarbon solvent is selected from the group consisting of squalane, C₃₂ alkane, C₃₂ isoalkane, C₅₄ alkane, C₅₄ isoalkane, and combinations thereof.

In an aspect, the solvent is a silicone solvent selected from the group consisting of dimethicone, phenyl dimethicone, caprylyl methicone, ethyl trisiloxane, cyclotetrasiloxane, cyclopentasiloxane, cyclohexasiloxane, and combinations thereof.

In an aspect, a defined amount of solvent is used in the preparation of the polyester elastomer. In an aspect, the amount of solvent is from 0% to 70% of the total weight of di-carboxylic acid or tri-carboxylic acid (A), mono-carboxylic acid (B), and polyol (C). In an aspect, the amount of solvent is from 0% to 50% of the total weight of di-carboxylic acid or tri-carboxylic acid (A), mono-carboxylic acid (B), and polyol (C). In an aspect, the amount of solvent is from 0% to 40% of the total weight of di-carboxylic acid or tri-carboxylic acid (A), mono-carboxylic acid (B), and polyol (C). In an aspect, the amount of solvent is from 0% to 30% of the total weight of di-carboxylic acid or tri-carboxylic acid (A), mono-carboxylic acid (B), and polyol (C). In an aspect, the range of solvent is from 0% to 20% of the total weight of di-carboxylic acid or tri-carboxylic acid (A), mono-carboxylic acid (B), and polyol (C). In an aspect, the amount of solvent is from 10% to 50% of the total weight of di-carboxylic acid or tri-carboxylic acid (A), mono-carboxylic acid (B), and polyol (C). In an aspect, the amount solvent is 50%, 40%, 30%, 20%, or 10% of the total weight of di-carboxylic acid or tri-carboxylic acid (A), mono-carboxylic acid (B), and polyol (C).

In an aspect, the amount of solvent is from 0% to 30% of the total weight of di-carboxylic acid or tri-carboxylic acid (A) and mono-carboxylic acid (B). In an aspect, the amount of solvent is from 0% to 20% of the total weight of di-carboxylic acid or tri-carboxylic acid (A) and mono-carboxylic acid (B). In an aspect, the amount of solvent is from 0% to 10% of the total weight of di-carboxylic acid or tri-carboxylic acid (A) and mono-carboxylic acid (B). In an aspect, the amount of solvent is from 10% to 30% of the total weight of di-carboxylic acid or tri-carboxylic acid (A) and mono-carboxylic acid (B). In an aspect, the solvent is 30%, 20%, or 10% of the total weight of di-carboxylic acid or tri-carboxylic acid (A) and mono-carboxylic acid (B).

In an aspect, no solvent is used to prepare the elastomer.

In an aspect, a solvent is used to prepare the polyester elastomer, which is removed after preparing the polyester elastomer to form a polyester elastomer powder. In an aspect, to the polyester elastomer powder a solvent/emollient can be added again to form a polyester elastomer gel, optionally applying a shear force as described below.

In an aspect, polyester elastomer is made from C₃₆ dimer acid, diglycerol, and isostearic acid with about 10% to about 40% by weight squalane based on the total weight of the polyester elastomer and the squalane. That is, in an aspect the invention relates to a composition of a polyester elastomer made from C₃₆ dimer acid, diglycerol, isostearic acid, and squalane, comprising about 10% to about 40% by weight squalane.

In an aspect, polyester elastomer is made from hydrogenated C₃₆ dimer acid, diglycerol, and oleic acid without any solvent or emollient.

### 6. Temperature

In an aspect, the method of preparing the elastomer comprises reacting at least one di-carboxylic acid or tri-carboxylic acid, at least one mono-carboxylic acid, and at least one polyol with mixing at a pre-determined temperature until an elastomer is formed. In an aspect, the method of preparing the elastomer comprises reacting at least one di-carboxylic acid or tri-carboxylic acid, at least one mono-carboxylic acid, at least one polyol, optionally at least one solvent or emollient, and optionally a catalyst with mixing at a pre-determined temperature until an elastomer is formed. In an aspect, the temperature range is from 30° C. to 250° C.

In an aspect, the reaction occurs at a temperature from about 30° C. to about 250° C. In an aspect, the reaction occurs at a temperature from about 60° C. to about 250° C. In an aspect, the reaction occurs at a temperature from about 30° C. to about 125° C. or about 40° C. to about 100° C. In an aspect, the reaction occurs at a temperature of about 30° C., about 35° C., about 40° C., about 45° C., about 50° C., about 55° C., about 60° C., about 65° C., about 70° C., about 75° C., about 80° C., about 85° C., about 90° C., about 95° C., about 100° C., about 105° C., about 110° C., about 115° C., about 120° C., about 125° C., about 130° C., about 135° C., about 140° C., about 145° C., about 150° C., about 155° C., about 160° C., about 165° C., about 170° C., about 175° C., about 180° C., about 185° C., about 190° C., about 195° C., about 200° C., about 205° C., about 210° C., about 215° C., about 220° C., about 225° C., about 230° C., about 235° C., about 240° C., about 245° C., or about 250° C.

### 7. Time

In an aspect, the reaction time is from about 12 hours to about 150 hours. In an aspect, the reaction time is from about 6 hours to about 24 hours. In an aspect, the reaction time is from about 8 hours to about 27 hours. In an aspect, the reaction time is about 6 hours, about 6.5 hours, about 7 hours, about 7.5 hours, about 8 hours, about 8.5 hours, about 9 hours, about 9.5 hours, about 10 hours, about 10.5 hours, about 11 hours, about 11.5 hours, about 12 hours, about 12.5 hours, about 13 hours, about 13.5 hours, about 14 hours, about 14.5 hours, about 15 hours, about 15.5 hours, about 16 hours, about 16.5 hours, about 17 hours, about 17.5 hours, about 18 hours, about 18.5 hours, about 19 hours, about 19.5 hours, about 20 hours, about 20.5 hours, about 21 hours, about 21.5 hours, about 22 hours, about 22.5 hours, about 23 hours, about 23.5 hours, about 24 hours, about 24.5 hours, about 25 hours, about 25.5 hours, about 26 hours, about 26.5 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 49 hours, about 50 hours, about 55 hours, about 60 hours, about 65 hours, about 70 hours, about 75 hours, about 80 hours, about 85 hours, about 90 hours, about 95 hours, about 100 hours, about 105 hours, about 110 hours, about 115 hours, about 120 hours, about 125 hours, about 130 hours, about 135 hours, about 140 hours, about 145 hours, or about 150 hours.

The reaction time can be adjusted by determination of the gel fraction achieved, and preferably the reaction time is such that the gel fraction of the polyester elastomer is greater than 60%. A method to measure the gel fraction in the polyester elastomer is described below.

### 8. By-Product Removal

In an aspect, the method further comprises removing water and alcohol by-product from the reaction. In a further aspect, the water and alcohol by-products are removed from the reaction by mixing and heating the reaction. In an aspect, the reaction is heated to above about 120° C. to remove the water and alcohol by-products. In an aspect, the water and alcohol by-products are removed from the reaction by nitrogen flow, by vacuum, or a combination thereof. In an aspect, water is removed by nitrogen stripping and vacuum, which have an impact on the reaction time.

### III. Method of Making a Polyester Elastomer Composition

### 1. Form

In an aspect, the polyester elastomer composition is a gel or a powder.

In an aspect, the polyester elastomer is processed into a gel as described herein.

### 2. Components

In an aspect, polyester elastomer is only comprised of crosslinked polyester without solvent or emollient. In an aspect, polyester elastomer is comprised of polyester without solvent or emollient. In an aspect, polyester elastomer is comprised of crosslinked polyester with solvent or emollient. In an aspect, polyester elastomer is comprised of polyester with solvent or emollient.

In an aspect, polyester elastomer composition is only comprised of polyester elastomer. In an aspect, polyester elastomer composition is comprised of polyester elastomer with solvent or emollient. In another aspect, polyester elastomer is comprised of polyester elastomer with more than one solvent or emollient. Solvents or emollients that can be used to prepare the polyester elastomer composition are described herein and can be selected from the solvents or emollients as defined herein. In another aspect, the polyester elastomer in polyester elastomer composition is in the range of 5 wt % to 100 wt %. In another aspect, the polyester elastomer in polyester elastomer composition is in the range of 5 wt % to 70 wt %. In another aspect, the polyester elastomer in polyester elastomer composition is in the range of 10 wt % to 60 wt %. In another aspect, the polyester elastomer in polyester elastomer composition is in the range of 20 wt % to 50 wt %.

In an aspect, polyester elastomer is only comprised of crosslinked polyester without any solvent or emollient. In such case it is usually a powder. In an aspect, polyester elastomer is comprised of crosslinked polyester with solvent or emollient. In another aspect, polyester elastomer is comprised of crosslinked polyester with more than one solvent or emollient. Solvents or emollients that can be used to prepare the polyester elastomer composition are described herein and can be selected from the solvents as defined herein. In another aspect, polyester elastomer is a powder.

In another aspect, the crosslinked polyester in polyester elastomer composition is in the range of 5 wt % to 50 wt %. In another aspect, the crosslinked polyester in polyester elastomer composition is in the range of 5 wt % to 30 wt %. In another aspect, the crosslinked polyester in polyester elastomer composition is in the range of 10 wt % to 30 wt %. Solvents or emollients that can be used to prepare the polyester elastomer composition are described herein and can be selected from the solvents or emollients as defined herein.

In an aspect, the polyester elastomer composition comprises at least one solvent or emollient added to the polyester elastomer during the shearing force process. Solvents or emollients that can be used to prepare the polyester elastomer composition are described herein and can be selected from the solvents as defined herein. In an aspect, the solvent or emollient is from about 20% to about 95% weight by weight of the composition. In an aspect, the solvent or emollient is from about 20% to about 50% weight by weight of the composition. In an aspect, the solvent or emollient is from about 50% to about 90% weight by weight of the composition. In an aspect, the solvent or emollient is from about 70% to about 90% weight by weight of the composition. In an aspect, the polyester elastomer composition comprises from about 50% to about 90% weight by weight of solvent or emollient, from about 50% to about 80% weight by weight of solvent or emollient, from about 50% to about 70% weight by weight of solvent or emollient, or from about 50% to about 60% weight by weight of solvent or emollient. In some embodiments, the polyester elastomer composition comprises about 20%, about 30%, about 40%, about 50%, about 60%, about 70%, about 80%, or about 90% weight by weight of solvent or emollient.

### 3. Process

In an aspect, the polyester elastomer obtained is crumbled or processed to form a polyester elastomer powder. In another aspect, the polyester elastomer is processed by a three roll mill to form a polyester elastomer powder.

The polyester elastomer composition can be also obtained as a powder if it still comprises a solvent or emollient added to the reaction mixture.

In an aspect, a composition is prepared by combining the polyester elastomer with one or more solvents or emollients to form a polyester elastomer gel.

In an aspect, polyester elastomer and solvent/emollient mixture are processed with a homogenizer to produce a gel optionally applying a shear force, e.g. by a high-shear disperser mixer.

In an aspect, a polyester elastomer is swelled in a solvent or emollient before being processed to make a gel at a temperature under 23° C. In an aspect, the time of polyester elastomer swelling in a solvent or emollient is from 1 hour to 1 week. In an aspect, the elastomer is subject to swelling in a solvent or emollient from 10 minutes to 1 week, from 10 minutes to 4 days, from 10 minutes to 3 days, from 10 minutes to 2 days, from 10 minutes to 1 day, from 10 minutes to 12 hours, from 10 minutes to 6 hours, from 10 minutes to 3 hours, from 10 minutes to 2 hours, from 10 minutes to 1 hour, or from 10 minutes to 30 minutes.

Once the initially produced polyester elastomer is prepared, it can be mixed with an additional quantity of at least one solvent or emollient that can be different from the solvent emollient used to prepare the initially produced elastomer. In some aspects, the at least one solvent or emollient used to prepare the elastomer is the same as the at least one solvent or emollient used to prepare the elastomer composition. The addition of an additional quantity of at least one solvent or emollient dilutes the gel composition and thereby adjusts its viscosity.

The present disclosure provides a method of preparing a polyester elastomer composition, wherein the method comprises:
(i) combining a polyester elastomer with at least one solvent or emollient thereby forming a swollen polymer elastomer; and
(ii) subjecting the swollen polyester elastomer to shear force thereby forming a polyester elastomer composition.

In an aspect, the polyester elastomer composition is a powder, a gel, or a paste.

In an aspect, the at least one solvent or emollient is selected from the solvents or emollients described herein.

### 4. Emollient

In an aspect, the preparation of the polyester elastomer gel occurs in the presence of an emollient. In an aspect, the emollient is a biobased or naturally derived. In an aspect, the emollient is a triglyceride emollient, a mono-ester emollient, a di-ester emollient, a citrate ester emollient, an ether emollient, a carbonate emollient, a hydrocarbon emollient, a silicone emollient, or a combination thereof.

In an aspect, the emollient is a triglyceride emollient of formula (IV)

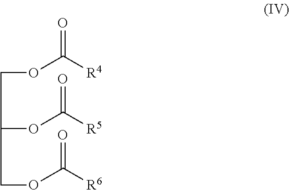

wherein
each R⁴, R⁵, and R⁶ are independently C₁-C₃₅ alkyl group, C₁-C₃₅ heteroalkyl group, C₂-C₃₅ alkene group, or C₂-C₃₅ heteroalkene group.

In an aspect, the emollient is of formula (IV), wherein R⁴, R⁵, and R⁶ are independently C₂-C₁₇ alkyl group or C₂-C₁₇ alkylene group.

In an aspect, the emollient is a triglyceride emollient selected from the group consisting of caprylic/capric triglyceride, triheptanoin, corn oil, soybean oil, olive oil, rape seed oil, cotton seed oil, coconut oil, almond oil, argon oil, rosehip oil, black seed oil, grape seed oil, avocado oil, apricot kernel oil, geranium oil, lavender oil, rosehip oil, macadamia oil, eucalyptus oil, sardine oil, herring oil, safflower oil, linseed oil, sunflower oil, olive oil, canola oil, sesame oil, cottonseed oil, palm oil, rapeseed oil, tung oil, fish oil, peanut oil, cuphea oil, milkweed oil, salicornia oil, whale oil, castor oil, and combinations thereof. In an aspect, the triglyceride emollient is selected from the group consisting of caprylic/capric triglyceride, triheptanoin, and combinations thereof.

In an aspect, the emollient is a mono-ester emollient of formula (V)

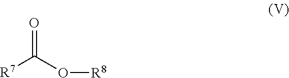

wherein
R⁷ is C₁-C₃₅ alkyl group, C₁-C₃₅ heteroalkyl group, C₂-C₃₅ alkene group, or C₂-C₃₅ heteroalkene group; and
R⁸ is C₁-C₃₅ alkyl group, C₁-C₃₅ heteroalkyl group, C₂-C₃₅ alkene group, or C₂-C₃₅ heteroalkene group.

In an aspect, the emollient is a mono-ester emollient of formula (V), wherein R⁷ is C₅-C₁₇ alkyl group or C₅-C₁₇ alkene group and R⁸ is C₂-C₁₇ alkyl group or C₂-C₁₇ alkene group.

In an aspect, the emollient is a mono-ester emollient selected from the group consisting of coco-caprylate, coco-caprate, jojoba oil, jojoba esters, isopropyl jojobate, ethyl macadamiate, isoamyl laurate, heptyl undecylenate, methylheptyl isostearate, isostearyl isostearate, glyceryl ricinoleate, isostearyl palmitate, myristyl myristate, octyldodecyl myristate, octyldodecyl hydroxystearate, butyl myristate, ethylhexyl cocoate, ethylhexyl palmitate, ethylhexyl stearate, butyl stearate, decyl oleate, isocetyl behenate, isocetyl myristate, isocetyl palmitate, isocetyl stearate, isodecyl oleate, isopropyl isostearate, isopropyl myristate, isopropyl palmitate, oleyl oleate, propylene glycol laurate, octyldodecyl erucate, C₁₂-C₁₃ alkyl lactate, C₁₂-C₁₅ alkyl lactate, isostearyl lactate, glycereth-5-lactate, lauryl lactate, myristyl lactate, oleyl lactate, laureth-2-benzoate, C₁₂-C₁₅ alkyl benzoate, C₁₂-C₁₅ pareth-3-benzoate, dipropylene glycol benzoate, isodecyl salicylate, C₁₂-C₁₅ alkyl salicylate, tridecyl salicylate, ethylhexyl isononanoate, cetyl ethylhexanoate, isononyl isononanoate, isodecyl ethylhexanoate, isodecyl isononanoate, tridecyl ethylhexanoate, isotridecyl isononanoate, isostearyl isononanoate, cetearyl isononanoate, laureth-2-ethylhexanoate, cetearyl ethylhexanoate, isodecyl neopentanoate, isostearyl neopentanoate, nyristyl neopentanoate, isostearyl behenate, octyldodecyl neopentanoate, tridecyl neopentanoate, and combinations thereof. In an aspect, the mono-ester emollient is selected from the group consisting of coco-caprylate/caprate, coco-caprylate, jojoba oil, isoamyl laurate, methylheptyl isostearate, C₁₂-C₁₃ alkyl lactate, C₁₂-C₁₅ alkyl lactate, lauryl lactate, ethylhexyl isononanoate, cetyl ethylhexanoate, isononyl isononanoate, isodecyl ethylhexanoate, isodecyl isononanoate, tridecyl ethylhexanoate, isotridecyl isononanoate, isostearyl isononanoate, cetearyl isononanoate, and combinations thereof. In an aspect, the mono-ester emollient is selected from the group consisting of coco-caprylate/caprate, coco-caprylate, isoamyl laurate, isononyl isononanoate, heptyl undecylenate, jojoba oil, jojoba esters, and combinations thereof.

In an aspect, the emollient is:
(a) a di-ester emollient of formula (VI)

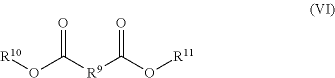

wherein
R⁹ is C₁-C₃₅ alkyl group, C₁-C₃₅ heteroalkyl group, C₂-C₃₅ alkene group, or C₂-C₃₅ heteroalkene group; and
R¹⁰ and R¹¹ are independently C₁-C₃₅ alkyl group, C₁-C₃₅ heteroalkyl group, C₂-C₃₅ alkene group, or C₂-C₃₅ heteroalkene group; or
(b) a di-ester emollient of formula (VII)

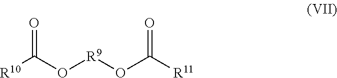

wherein
R⁹ is C₁-C₃₅ alkyl group, C₁-C₃₅ heteroalkyl group, C₂-C₃₅ alkene group, or C₂-C₃₅ heteroalkene group; and
R¹⁰ and R¹¹ are independently H, C₁-C₃₅ alkyl group, C₁-C₃₅ heteroalkyl group, C₂-C₃₅ alkene group, or C₂-C₃₅ heteroalkene group; or
(c) a di-ester emollient of formula (VIII)

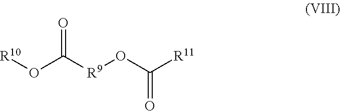

wherein
R⁹ and R¹⁰ are independently C₁-C₃₅ alkyl group, C₁-C₃₅ heteroalkyl group, C₂-C₃₅ alkene group, or C₂-C₃₅ heteroalkene group; and
R¹¹ is H, C₁-C₃₅ alkyl group, C₁-C₃₅ heteroalkyl group, C₂-C₃₅ alkene group, or C₂-C₃₅ heteroalkene group.

In an aspect, the emollient is a di-ester emollient of formula (VI), formula (VII), or formula (VIII), wherein R¹¹ is C₂-C₁₀ alkyl group or C₂-C₁₀ alkene group and R⁹ and R¹⁰ are independently C₁-C₁₂ alkyl group or C₂-C₁₂ alkene group.

In an aspect, the di-ester emollient is selected from the group consisting of diethyl succinate, dibutyl succinate, diethyhexyl succinate, diisopropyl sebacate, dimethyl sebacate, diethyl sebacate, dibutyl sebacate, diisostearyl dimer, diisostearyl malate, isostearyl stearoyl stearate, isocetyl stearoyl stearate, octyldodecyl stearoyl stearate, diethylhexyl malate, diethylhexyl maleate, dipropylene glycol dibenzoate, dicapryl adipate, dicaprylyl maleate, diisopropyl dimer, diisopropyl adipate, diisobutyl adipate, diisopropyl sebacate, diisostearyl dimer, diethyhexyl succinate, diethylene glycol diethylhexanoate, neopentyl glycol dicaprate, propylene glycol dicaprylate/dicaprate, neopentyl glycol diisostearate, neopentyl glycol diethylhexanoate, neopentyl glycol diheptanoate, and combinations thereof. In an aspect, the di-ester emollient is selected from the group consisting of dicapryl adipate, dicaprylyl maleate, diisopropyl adipate, diisobutyl adipate, diethyl succinate, dibutyl succinate, diethyhexyl succinate, diisopropyl sebacate, dimethyl sebacate, diethyl sebacate, dibutyl sebacate, neopentyl glycol diethylhexanoate, neopentyl glycol diheptanoate, and combinations thereof.

In an aspect, the emollient is a citrate ester emollient of formula (IX)

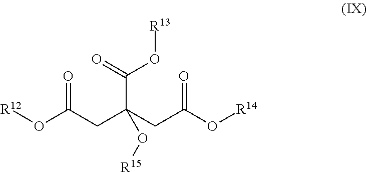

wherein
R¹², R¹³, R¹⁴, and R¹⁵ are independently H, C₁-C₃₅ alkyl group, C₁-C₃₅ heteroalkyl group, C₂-C₃₅ alkene group, or C₂-C₃₅ heteroalkene group, wherein at least one of R¹², R¹³, R¹⁴, and R¹⁵ is not H.

In an aspect, the emollient is a citrate ester emollient of formula (IX), wherein R¹², R¹³, and R¹⁴ are independently C₁-C₁₀ alkyl group or C₂-C₁₀ alkene group and R¹⁵ is an acetyl group.

In an aspect, the emollient is a citrate ester emollient selected from the group consisting of tricaprylyl citrate, triisostearyl citrate, triisocetyl citrate, trioctyldodecyl citrate, triethyl citrate, tributyl citrate, acetyl triethyl citrate, acetyl tributyl citrate, trioctyldodecyl citrate, triisocetyl citrate, and combinations thereof.

In an aspect, the emollient is an ether emollient of formula (X)

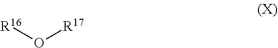

wherein
R¹⁶ and R¹⁷ are independently H, C₂-C₂₀ alkyl group, C₂-C₂₀ heteroalkyl group, C₂-C₂₀ alkene group, or C₂-C₂₀ heteroalkene group, wherein at least one of R¹⁶ and R¹⁷ is not H.

In an aspect, the emollient is an ether emollient of formula (X), wherein R¹⁶ and R¹⁷ are independently C₂-C₂₀ alkyl group.

In an aspect, the emollient is an ether emollient selected from the group consisting of dicaprylyl ether, didecyl ether, panthenyl ethyl ether, dicetyl ether, dimyristyl ether, distearyl ether, dilauryl ether, and combinations thereof. In an aspect, the ether emollient is selected from the group consisting of dicaprylyl ether, didecyl ether, and combinations thereof.

In an aspect, the emollient is a carbonate emollient of formula (XI)

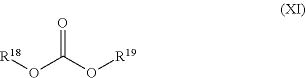

wherein
R¹⁸, and R¹⁹ are independently H, C₂-C₂₀ alkyl group, C₂-C₂₀ heteroalkyl group, C₂-C₂₀ alkene group, or C₂-C₂₀ heteroalkene group.

In an aspect, the emollient is a carbonate emollient of formula (XI), wherein R¹⁸ and R¹⁹ are independently C₂-C₂₀ alkyl group.

In an aspect, the emollient is a carbonate emollient selected from the group consisting of dicaprylyl carbonate, diethyl hexyl carbonate, and combinations thereof.

In an aspect, the emollient is a hydrocarbon with number of carbon atoms from C₄ to C₆₀. In an aspect, the emollient is a hydrocarbon with number of carbon atoms from C₁₀ to C₅₀. In an aspect, the emollient is a hydrocarbon with number of carbon atoms from C₂₀ to C₄₀.

In an aspect, the emollient is a hydrocarbon emollient selected from the group consisting of farnesene, hydrogenated farnesene, coconut alkanes, coconut/palm kernel alkanes, C₉-C₁₂ alkane, C₁₀-C₁₃ alkane, C₁₂-C₁₇ alkane, C₁₃-C₁₄ alkane, C₁₃-C₁₅ alkane, C₁₄-C₁₇ alkane, C₁₄-C₁₉ alkane, C₁₄-C₂₀ alkane, C₁₄-C₂₂ alkane, C₁₅-C₁₉ alkane, C₂₁-C₂₈ alkane, C₁₇-C₂₃ alkane, C₉-C₁₂ isoalkane, C₉-C₁₃ isoalkane, C₉-C₁₄ isoalkane, C₉-C₁₆ isoalkane, C₁₀-C₁₁ isoalkane, C₁₀-C₁₂ isoalkane, C₁₀-C₁₃ isoalkane, C₁₁-C₁₂ isoalkane, C₁₁-C₁₃ isoalkane, C₁₁-C₁₄ isoalkane, C₁₂-C₁₄ isoalkane, C₁₂-C₁₅ isoalkane, C₁₂-C₂₀ isoalkane, C₁₃-C₁₄ isoalkane, C₁₃-C₁₆ isoalkane, C₁₄-C₁₆ isoalkane, C₁₅-C₁₉ isoalkane, C₁₀-C₁₆ olefin, C₁₂-C₁₈ olefin, C₁₈-C₂₆ olefin, C₂₀ olefin, C₂₀-C₂₄ olefin, C₂₄-C₃₀ olefin, C₂₆-C₂₈ olefin, C₂₆-C₅₄ olefin, C₂₈-C₃₆ olefin, C₂₈-C₅₂ olefin, C₃₀-C₃₈ olefin, C₃₀-C₄₅ olefin, C₄-C₁₂ olefin, C₄. C₆ olefin, C₅-C₆ olefin, hydrogenated poly(C₆/C₁₀/C₁₄ olefin), hydrogenated poly(C₆-C₁₂ olefin), hydrogenated poly(C₆-C₁₄ olefin), hydrogenated poly(C₆-C₂₀ olefin), hydrogenated poly(C₈/C₁₂ olefin), poly(C₂₀-C₂₈ olefin), poly(C₃₀-C₄₅ olefin), poly(C₄-C₁₂ olefin), poly(C₆-C₁₄ olefin), hexadecene, C₃₂ alkane, C₃₂ isoalkane, C₅₄ alkane, C₅₄ isoalkane, diethylhexylcyclohexane, undecane, tridecane, tetradecane, pentadecane, hexadecane, octadecane, docosane, squalane, hydrogenated polyisobutene, polybutene, hydrogenated polydecene, hydrogenated didecene, mineral oil, liquidum, petrolatum, dodecane, isohexadecane, isododecane, isoeicosane, and combinations thereof. In an aspect, the hydrocarbon emollient is selected from the group consisting of squalane, farnesene, hydrogenated farnesene, coconut alkanes, C₉-C₁₂ alkane, C₁₃-C₁₅ alkane, C₁₄-C₁₉ alkane, C₁₄-C₂₀ alkane, C₁₄-C₂₂ alkane, C₁₅-C₁₉ alkane, C₁₃-C₁₆ isoalkane, dodecane, undecane, tridecane, tetradecane, pentadecane, hexadecane, hexadecene, octadecane, squalane, isododecane, isohexadecane, C₃₂ alkane, C₃₂ isoalkane, C₅₄ alkane, C₅₄ isoalkane, and combinations thereof. In an aspect, the hydrocarbon emollient is selected from the group consisting of squalane, hydrogenated farnesene, coconut alkanes, C₉-C₁₂ alkane, C₁₃-C₁₅ alkane, C₁₃-C₁₆ isoalkane, C₁₄-C₁₉ alkane, dodecane, tetradecane, isododecane, hexadecane, octadecane, hexadecene, C₃₂ alkane, C₃₂ isoalkane, C₅₄ alkane, C₅₄ isoalkane, and combinations thereof. In an aspect, the hydrocarbon emollient is selected from the group consisting of squalane, C₃₂ alkane, C₃₂ isoalkane, C₅₄ alkane, C₅₄ isoalkane, and combinations thereof.

In an aspect, the emollient is a silicone emollient selected from the group consisting of dimethicone, phenyl dimethicone, caprylyl methicone, ethyl trisiloxane, cyclotetrasiloxane, cyclopentasiloxane, cyclohexasiloxane, and combinations thereof

### 5. Polyester Elastomer

In an aspect, the solubility of polyester elastomer is measured by mixing 1 gram of polyester elastomer with 100 gram of test solvent with magnetic stirring in a sealed glass container for 24 hours under room temperature (23° C.). Afterward the polyester elastomer and test solvent is passed through filtration and the solid on filter is obtained and dried under 80° C. for 20 hours or dried to constant weight, optionally in vacuo. The dried solid is considered as the fraction of polyester elastomer which is not soluble in the test solvent.

In an aspect, the fraction of the polyester elastomer which is not soluble in the ethyl acetate by weight is greater than or equal to 40% of the total weight of the polyester elastomer.

In another aspect, the fraction of the polyester elastomer which is not soluble in ethyl acetate is 40% to 90% of the total weight of the polyester elastomer. In another aspect, the fraction of the polyester elastomer which is not soluble in ethyl acetate is 50% to 90% of the total weight of the polyester elastomer.

A sample of polyester elastomer without solvent or emollient, or a sample of polyester elastomer with solvent or emollient can be used. In case a sample of polyester elastomer with solvent or emollient is used, the weight of the solvent or emollient in the composition is considered and subtracted from the total composition weight to determine the weight percentage of the fraction of the polyester elastomer which is not soluble in the ethyl acetate.

The method of Soxhlet extraction can be used to determine the % by weight of crosslinked polyester (gel fraction) in the polyester elastomer. The basis for the test is to extract soluble low Mw components (and optionally present solvents or emollients) from the high Mw and crosslinked insoluble components through an extraction test. The percentage of the crosslinked polyester is defined as the ratio of the weight of insoluble residue (dried gel) to the initial weight of the polyester elastomer sample.

The gel fraction of the polyester elastomers, defined as

$$\\ Gel fraction ( \% ) = 100 \times \frac{weight of dried gel ( insoluble residue of the extraction )}{total weight of the polyester elastomer used in the extraction}$$

is determined as follows.

A sample of polyester elastomer without solvent or emollient, or a sample of polyester elastomer with solvent or emollient can be used. In case a sample of polyester elastomer with solvent or emollient is used, the weight of the solvent or emollient in the polyester elastomer is considered and subtracted from the sample weight to determine the total weight of the polyester elastomer used in the extraction in the formula above.

A cellulose extraction thimble is weighed. 3.0-3.5 grams of a sample of a polyester elastomer is weighed and placed in the cellulose extraction thimble. About 125-150 mL of ethyl acetate (EtOAc) are placed in a 250 mL round bottom flask. The thimble with the sample is placed into the Soxhlet extraction column. The EtOAc solution is heated to 80° C. (Reflux, bp 77° C.) and maintained at reflux for 1 hour to extract the polymer sample solubles and the optional present solvent or emollient. After 2 hours the EtOAc solution is allowed to cool to room temperature (about 23° C.). The thimble containing the polymer residue is removed from the Soxhlet apparatus. The thimble is placed in a desiccator under vacuum, a vacuum oven, or a vented oven (50° C.) to remove the residual EtOAc (24 hours). After the EtOAc has been removed, the weight of the thimble containing the dried gel is determined. The weight of the dried gel is calculated by subtraction of the weight of the cellulose extraction thimble. The gel fraction is calculated from the above equation. Any amount of solvent or emollient in the sample is subtracted from the “total weight of the polyester elastomer used in the extraction”.

In an aspect, the gel fraction of the polyester elastomer is greater than 20%. In an aspect, the gel fraction of the polyester elastomer is greater than 40%. In an aspect, the gel fraction of the polyester elastomer is greater than 50%. In an aspect, the gel fraction of the polyester elastomer is greater than 60%. In an aspect, the gel fraction of the polyester elastomer is greater than 70%.

Swelling test for the polyester elastomer is to determine the swelling capacity of the polyester elastomer through a weight of solvent or emollient retained by the polyester elastomer. The swelling ratio (SR—sometimes called swelling value) is determined according to the equation:

$$\\ SR = \frac{Ws - Wi}{Wi}$$

where:
Ws is the weight of the swollen polyester elastomer, and
Wi is the weight of the initial (dry polymer).

The swelling ratio of the polyester elastomer is suitably determined as follows.

A sample of polyester elastomer without solvent or emollient, or a sample of polyester elastomer with solvent or emollient can be used. In case a sample of polyester elastomer with solvent or emollient is used, the weight of the solvent or emollient already present in the composition is considered and subtracted from the initial sample weight Wi.

The swelling procedure is carried out at ambient temperature (23° C.).

About 1.9-2.1 grams of the polyester elastomer is placed in a 25 mL beaker. In the same beaker the polyester elastomer is mixed with 24.9-25.1 grams of coco-caprylate/caprate as solvent. The polyester elastomer is allowed to disperse and absorb (swell) the solvent for 30 minutes. The weight of the filter component (such as Thermo Scientific™ Nalgene™ Rapid-Flow™ Sterile Disposable Filter Units) is determined. After the polyester elastomer has swelled, the mixture in the beaker is mixed and poured into the filter. The beaker is rinsed with about 4.9-5.1 grams of coco-caprylate/caprate solvent to complete transfer of the swelled polyester elastomer. The excess solvent in the gel mixture is allowed to pass through the filter. The filter with swollen polyester elastomer is weighed when no excess solvent is observed on its surface (which can take about 4-18 hours) to give Ws.

The swelling ratio (SR) is calculated by the equation above.

In an aspect, the swelling ratio of the elastomer is from about 1 gram/gram to about 15 gram/gram, from about 1 gram/gram to about 5 gram/gram, from about 1 gram/gram to about 4 gram/gram, from about 1 gram/gram to about 2 gram/gram. In some embodiments, the swelling value of the elastomer is about 15 gram/gram, about 14 gram/gram, about 13 gram/gram, about 12 gram/gram, about 11 gram/gram, about 10 gram/gram, about 9 gram/gram, about 8 gram/gram, about 7 gram/gram, about 6 gram/gram, 5 gram/gram, about 4.8 gram/gram, about 4.6 gram/gram, about 4.4 gram/gram, about 4.2 gram/gram, about 4 gram/gram, about 3.8 gram/gram, about 3.6 gram/gram, about 3.4 gram/gram, about 3.2 gram/gram, about 3 gram/gram, about 2 gram/gram, or about 1 gram/gram.

FIG. 2 is a bar graph showing the swelling value of polyester elastomers prepared with 4 different ratios of monomers A (C₃₆ dimer acid), B (oleic acid), and C (diglycerol). The molar ratio of B to C was kept constant. The elastomers were swelled in coco-caprylate/caprate solvent for 18 hours under 22° C. As seen in FIG. 2, the ratio of A/C has a profound impact on the swelling value of the polyester elastomer synthesized. The lowest swelling value of the polyester elastomer occurs between the molar ratio of A/B/C of 1.5/0.5/1 and 2/0.5/1.

FIG. 4 is a bar graph showing the swelling value of elastomers prepared with 4 different ratios of monomers A (C₃₆ dimer acid), B (oleic acid), and C (diglycerol). The molar ratio of A to C was kept constant. The elastomers were swelled in coco-caprylate/caprate emollient for 18 hours under 22° C. As seen in FIG. 4, the swelling value of the polyester elastomer increases with increasing molar ratio of B/C.

### 6. Polyester Elastomer Gel

In an aspect, a polyester elastomer composition is prepared by shearing the polyester elastomer with a solvent or emollient, as described herein, to form a sheared polyester elastomer gel. In another aspect, a polyester elastomer gel is prepared by combining the polyester elastomer, as described herein, with a solvent or emollient, as described herein, thereby forming a mixture and shearing the mixture.

In an aspect, the shear force is provided by any type of mixing and shearing equipment. In an aspect, the mixing and shearing equipment is batch mixer, planetary mixer, single or multiple screw extruder, dynamic or static mixer, colloid mill, homogenizer, sonolator, three roll mill, or a combination thereof.

Subjecting these compositions to a shearing force produces a polyester elastomer gel suitable for use in personal care or cosmetic applications that has an improved spreadability and an improved substance or feel. The personal care applications where this property is most desirable include, but is not limited to, use in deodorants, antiperspirants, skin creams, facial creams, hair care products such as shampoos, mousses, and styling gels, protective creams, color cosmetics such as lipsticks, foundations, blushes, makeup, and mascara, and other cosmetic formulations.

In an aspect, the viscosity of the polyester elastomer gel is from about 10 cp to about 1,000,000 cp as measured by rheometer at a shear rate of 0.1 s⁻¹. In an aspect, the viscosity of the gel at 25° C. is from about 30,000 cp to about 900,000 cp. In an aspect, the viscosity of the gel is about 10 cp, about 1,000 cp, about 5,000 cp, about 10,000 cp, about 15,000 cp, about 20,000 cp, about 25,000 cp, about 30,000 cp, about 35,000 cp, about 40,000 cp, about 45,000 cp, about 50,000 cp, about 55,000 cp, about 60,000 cp, about 65,000 cp, about 70,000 cp, about 75,000 cp, about 80,000 cp, about 85,000 cp, about 90,000 cp, about 95,000 cp, about 100,000 cp, about 150,000 cp, about 200,000 cp, about 250,000 cp, about 300,000 cp, about 350,000 cp, about 400,000 cp, about 450,000 cp, about 500,000 cp, about 550,000 cp, about 600,000 cp, about 650,000 cp, about 700,000 cp, about 750,000 cp, about 800,000 cp, about 850,000 cp, about 900,000 cp, about 950,000 cp, or about 1,000,000 cp.

The viscosity of the polyester elastomer gel is measured by Anton Paar rheometer MCR 301, with probe PP25/S at gap 1 mm. The measuring profile is to use flow curve with shear rate 0.01-100/s under temperature 25° C. In measurement, a sample is loaded onto the rheometer stage, the probe is lowered, and the sample is allowed to equilibrate for 3 minutes, after which the test is performed. The viscosity at 10/s is reported.

In an aspect, the polyester elastomer gel is comprised of particles of size from about 1 μm to about 500 μm as measured by a laser diffraction particle size analyzer. In an aspect, the gel is comprised of particles of size from about 20 μm to about 400 μm. In an aspect, the gel is comprised of particles of size of about 1 μm, about 5 μm, about 10 μm, about 15 μm, about 20 μm, about 25 μm, about 30 μm, about 35 μm, about 40 μm, about 45 μm, about 50 μm, about 75 μm, about 100 μm, about 125 μm, about 150 μm, about 175 μm, about 200 μm, about 225 μm, about 250 μm, about 275 μm, about 300 μm, about 325 μm, about 350 μm, about 375 μm, or about 400 μm.

In an aspect, the polyester elastomer gel is comprised of particles of size with D10 between 10 μm to 50 μm, D50 between 20 μm to 100 μm, and D90 between 30 μm to 200 μm.

The size of particles in polyester elastomer gel is measured by HORIBA Scientific Partica LA-960 Laser Scattering Particle Size Analyzer. The sample is prepared by blending 0.3 g of elastomer gel with 15 g of coco-caprylate/caprate and mixing thoroughly at 23° C. A few drops of the diluted gel sample are transferred into the cuvette containing neat coco-caprylate/caprate while constantly stirring. Once the transmittance reaches the acceptable range, the measurement is performed at 23° C. Particle size values of D10, D50 and D90 are reported. The parameter D10 signifies the point in the size distribution, up to and including which, 10% of the total volume of material in the sample is ‘contained’. The parameter D50 signifies the point in the size distribution, up to and including which, 50% of the total volume of material in the sample is ‘contained’. The parameter D90 signifies the point in the size distribution, up to and including which, 90% of the total volume of material in the sample is ‘contained’.

Polyester elastomer gel according to the present invention are characterized by the oscillation amplitude as well as oscillation frequency-dependent rheology tests at 25° C. In the linear viscoelastic region within the frequency range from 0.01-100 Hz, a gel has a storage modulus G′ which is always greater than the loss modulus G″. G′ and G″ here are rheological parameters known to the person skilled in the art. The elastic or storage modulus, designated as G′, is an indicator of how elastic the material is i.e., how much mechanical energy is being stored per cycle of deformation whereas, the viscous or loss modulus, namely G″, is the measure of the lost or dissipated mechanical energy as heat and/or other form per cycle of deformation and they collectively quantify the elastic or viscous fraction of viscoelastic solids and/or liquids and are described for example in Ferry, J. D., Viscoelastic Properties of Polymers, John Wiley & Sons, Inc. New York, 1980.

The polyester elastomer gels according to the invention have an excellent yield point which, for example, has an advantageous effect on their thickening properties and also their ability to stabilize dispersed constituents of personal care formulations. For dynamic oscillation rheology tests, for example a MCR 301 Rheometer (Anton Paar, Graz, Austria) equipped with a 25 mm parallel plate steel geometry can be used.

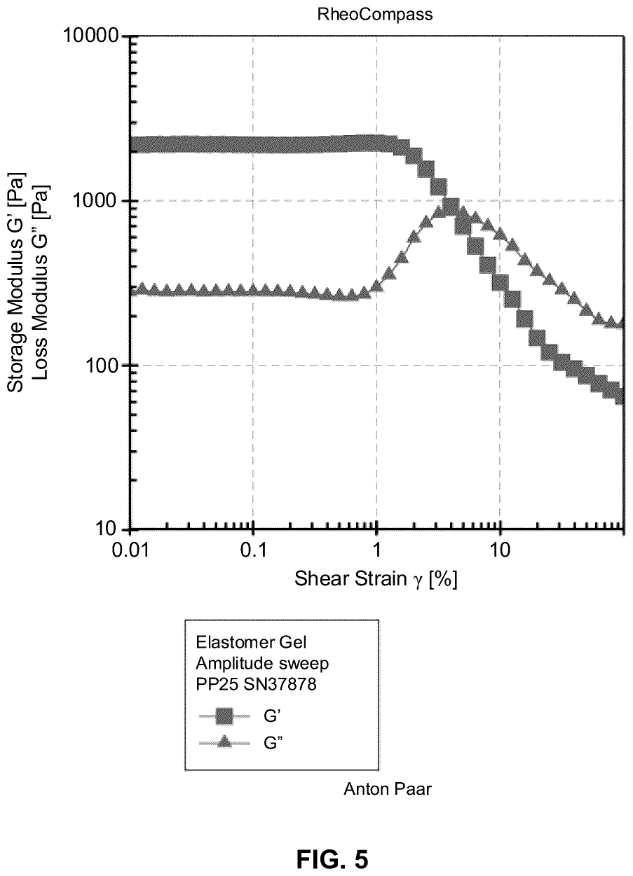

FIG. 5 is a line graph showing the rheology profile of the polyester elastomer gel prepared by processing the polyester elastomer of Example 1 with coco-caprylate/caprate emollient.

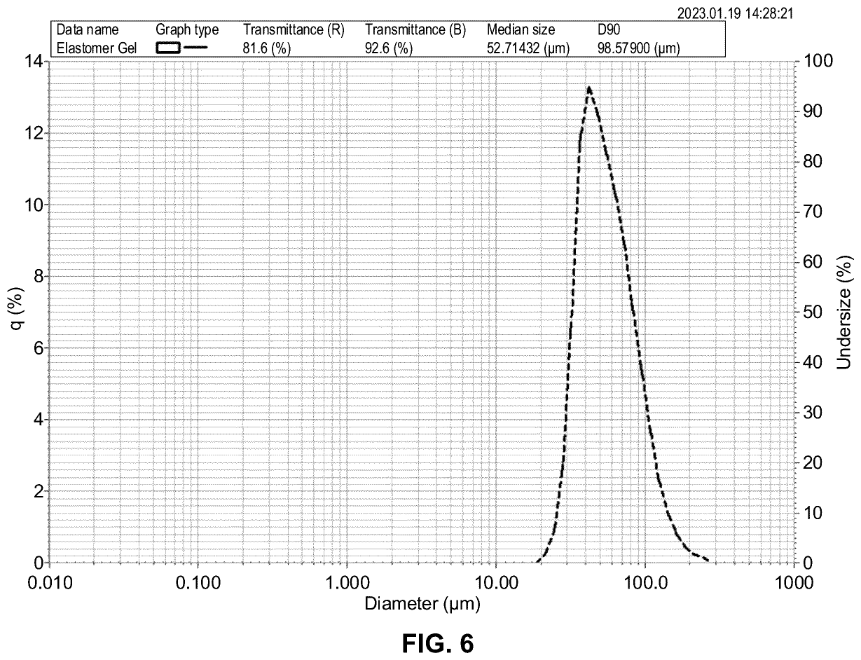

FIG. 6 is a line graph showing the particle distribution of the polyester elastomer gel prepared by processing the polyester elastomer of Example 1 with coco-caprylate/caprate emollient.

Polyester elastomer gels are notable for the fact that, at a shear rate of 1 l/s and a temperature of 25° C., they have a viscosity of less than 100,000,000 cp and at the same time satisfies G′>G″; Tan-δ<1 within the linear viscoelastic region demonstrating a frequency nearly invariant characteristics. The polyester gels prepared by the methods described herein are characterized by good flowability, which has an advantageous effect on their handleability and processability, but nevertheless have a pronounced yield point and therefore good thickening and stabilizing properties.

In an aspect, the storage modulus (G′) of the gel is from about 10 Pa to about 100,000 Pa as measured by rheometer within linear viscoelastic region using dynamic rheology. In an aspect, the storage modulus (G′) of the gel is from about 100 Pa to about 50,000 Pa. In an aspect, the storage modulus (G′) of the gel is from about 500 Pa to about 30,000 Pa, In an aspect, the storage modulus (G′) of the gel is about 10 Pa, about 100 Pa, about 500 Pa, about 700 Pa, about 800 Pa, about 1,000 Pa, about 1,500 Pa, about 2,000 Pa, about 2,500 Pa, about 5,000 Pa, about 10,000 Pa, about 15,000 Pa, about 25,000 Pa, about 50,000 Pa, or about 100,000 Pa.

In an aspect, the loss modulus (G″) of the gel is from about 10 Pa to about 100,000 Pa as measured by rheometer within linear viscoelastic region using dynamic rheology. In an aspect, the loss modulus (G″) of the gel is from about 100 Pa to about 50,000 Pa. In an aspect, the loss modulus (G″) of the gel is from about 500 Pa to about 30,000 Pa, In an aspect, the loss modulus (G″) of the gel is about 10 Pa, about 100 Pa, about 500 Pa, about 700 Pa, about 800 Pa, about 1,000 Pa, about 1,500 Pa, about 2,000 Pa, about 2,500 Pa, about 5,000 Pa, about 10,000 Pa, about 15,000 Pa, about 25,000 Pa, about 50,000 Pa, or about 100,000 Pa.

The storage modulus G′ and loss modulus G″ of the polyester elastomer gel are measured by Anton Paar rheometer MCR 301, with probe PP25/S at gap 1 mm. The measuring profile is to use flow curve with shear rate 0.01-100/s under temperature 25° C. The measuring profile is to use amplitude sweep with oscillatory strain 0.001-100%, with frequency 1 Hz and under temperature 25° C. In measurement sample is loaded onto the rheometer stage, the probe is lowered and the sample is allowed to equilibrate for 3 minutes after which the amplitude sweep test is performed. The LVR region is determined and the respective G′ value is reported.

In an aspect, the polyester elastomer composition is prepared using the methods described herein.

In an aspect, the polyester elastomers described herein are produced using the principles of green chemistry. In an aspect, the polyester elastomers described herein are produced by a simple, efficient environmentally friendly process, with no toxic raw materials used, and no toxic side products generated.

In an aspect, the polyester elastomer gels described herein are produced using the principles of green chemistry. In an aspect the polyester elastomer gels described herein are produced by a simple, efficient environmentally friendly process, with no toxic raw materials used, and no toxic side products generated.

### IV. Personal Care Formulations

In an aspect of the present disclosure, the polyester elastomers described herein are incorporated into a personal care formulation. In an aspect, the polyester elastomer gels prepared from the elastomers described herein are incorporated into a personal care formulation.

In an aspect, the polyester elastomer gel has excellent properties including clarity, thixotropy, shear thinning, and spread smoothly on the skin. In an aspect, the polyester gels are applied to cosmetic and medical products as the base oil or as key ingredient.

In an aspect, the personal care formulation further comprises a preservative, an antioxidant, a chelating agent, a gum or thickener, an oil, a wax, a fragrance, an essential oil, an emulsifier, a surfactant, or combinations thereof.

In an aspect, the personal care formulation is a deodorant, an antiperspirant, a skin cream, a facial cream, a hair shampoo, a hair conditioner, a mousse, a hair styling gel, a hair spray, a protective cream, a lipstick, a facial foundations, blushes, makeup, a mascara, a skin care lotion, a moisturizer, a facial treatment, a personal cleanser, a facial cleanser, a bath oil, a perfume, a shaving cream, a pre-shave lotion, an after-shave lotion, a cologne, a sachet, or a sunscreen.

In an aspect, the polyester elastomer is capable of being crumbled to form an elastomer powder. In an aspect, the polyester elastomer powder has the unique property of being easily rubbed-in on the skin or provides certain sensory benefit in personal care formulations. In an aspect, the polyester elastomer is used in solid cosmetics such as in antiperspirants and deodorants.

### V. Use of the Gels

In an aspect, the disclosure provides to the use of a polyester elastomer gel composition described herein for personal care formulations.

### EXAMPLES

The following examples are included to demonstrate various aspects of the present disclosure. It should be appreciated by those of skill in the art that the techniques disclosed in the examples that follow represent techniques discovered by the inventors to function well in the practice of the disclosure, and thus can be considered to constitute preferred modes for its practice. However, those of skill in the art should, in light of the present disclosure, appreciate that many changes can be made in the specific examples which are disclosed and still obtain a like or similar result without departing from the spirit and scope of the disclosure.

### Example 1: Preparation of Polyester Elastomer and Polyester Elastomer Gel

In a suitable vessel equipped with agitation, heat, and an ability to distill off water, 120 g hydrogenated C₃₆ dimer acid and 4 g oleic acid were added along with 20 g diglycerol. Next 80 g squalane was added as emollient. Next, 1 g of methanesulfonic acid was added. After all ingredients had been charged under agitation, the temperature was raised to 120° C., and water was stripped off as formed. The temperature was held 14-18 hours or until gelation took place and polymer elastomer was formed. Thereafter the elastomer was broken into a powder by mechanical stirring.

The 100 g polyester elastomer was also processed using 160 g coco-caprylate/caprate as emollient to form a polyester elastomer gel by rotor stator homogenizer. The rheology profile of the polyester gel was measured using a rheometer within linear viscoelastic region with dynamic rheology. As shown in FIG. 5, the polyester gel had a storage modulus (G′) of about 2000 Pa and a loss modulus (G″) of about 300 Pa. The size of the particles in the polyester gel was measured by laser diffraction. As shown in FIG. 6, the polyester gel was comprised of particles with a median particle size of 53 μm.

### Example 2: Preparation of Polyester Elastomer

In a suitable vessel equipped with agitation, heat, and an ability to distill off water, 120 g hydrogenated dimer acid and 6 g isostearic acid were added along with 25 g polyglycerol-3. After all ingredients had been charged under agitation, the temperature was raised to 140° C., and water was stripped off as formed. The temperature was held 25-30 hours or until gelation took place and polymer elastomer was formed. Thereafter the elastomer was broken into a powder by mechanical stirring.

### Example 3: Preparation of Polyester Elastomer and Polyester Elastomer Gel

In a suitable vessel equipped with agitation, heat, and an ability to distill off water, 175 g hydrogenated dimer acid and 12 g isostearic acid were added along with 35 g diglycerol. Next, 40 g of squalane was added as emollient. After all ingredients had been charged under agitation, the temperature was raised to 200° C., and water was stripped off as formed. The temperature was held 20-30 hours or until gelation took place and polymer elastomer was formed. Thereafter, the elastomer was broken into a powder by mechanical stirring. The gel fraction was 76% and was measured by the method of Soxhlet extraction described herein. The swelling ratio in coco-caprylate/caprate was 4.8.

100 g polyester elastomer was also processed with 200 g coco-caprylate/caprate as emollient to form a polyester gel.

### Example 4: Cosmetic Compositions

### Preparation of an Anhydrous Primer

Mixed the ingredients in Phase A of Table 1 together in a beaker until homogenous. Slowly added the ingredients in Phase B of Table 1 to the beaker and mixed until homogenous. See formulation specifics in Table 1.

TABLE 1

| Phase | Ingredient | INCI Name | % W/W |
|---|---|---|---|
| A | Polyester Elastomer Gel | | 66.00 |
| | according to the invention | | |
| | LexFeel Natural | Heptyl | 29.00 |
| | | Undecylenate | |
| B | Rice Silk SN | Oryza | 5.00 |
| | | Sativa Hull Powder | |

### Example 5: Preparation of an O/W Skin Cream

Combined all of the ingredients in Phase A of Table 2 together in a beaker and heated to 65° C. Mixed until homogenous. In a separate beaker, combined all of the ingredients in Phase B of Table 2 together and heat to 65° C. Mixed until homogenous. Once both phases were at a temperature of 65° C., slowly added Phase B to Phase A under homogenization. Homogenized for 5 minutes. Slowly mixed to cool. See formulation specifics in Table 2.

TABLE 2

| | | | % |
|---|---|---|---|
| Phase | Ingredient | INCI Name | W/W |
| A | DI Water | Aqua | 64.55 |
| | Keltrol CG-SFT | Xanthan Gum | 0.20 |
| | Sodium Chloride | Sodium Chloride | 0.50 |
| | Glycerin | Glycerin | 1.20 |
| | Spectrastat G2 | Caprylhydroxamic Acid | 1.00 |
| | Natural MB | (and) Glyceryl Caprylate | |
| | | (and) Glycerin | |
| | Dissolvine ® NA2-S | Disodium EDTA | 0.05 |
| B | Emulgin SG | Sodium Stearoyl Glutamate | 0.50 |
| | Vercarem GEC | Glyceryl Stearate Citrate | 4.00 |
| | Lanette O | Cetearyl Alcohol | 3.00 |
| | MCT Oil | Caprylic/Capric Triglyceride | 5.00 |
| | LexFeel Natural | Heptyl Undecylenate | 10.00 |
| | Polyester Elastomer | | 10.00 |
| | Gel according to the | | |
| | invention | | |

### Example 6: Preparation of a Sun Serum

Combined all of the ingredients in Phase A of Table 3 together in a beaker. Heated up to 75 PC and mixed until homogenous. Once everything had evenly dispersed, continued to mix for 30 minutes. Then slowly added the ingredients in Phase B of Table 3 one by one and mixed until homogenous. Let cool at room temperature overnight. It took a few hours for the formulation to fully set up. See formulation specifics in Table 3.

TABLE 3

| | | | % |
|---|---|---|---|
| Phase | Ingredient | INCI Name | W/W |
| A | Avobenzone | Avobenzone | 3.00 |
| | Parsol EHS | Ethylhexyl Salicylate | 5.00 |
| | Parsol 340 | Octocrylene | 10.00 |
| | Parsol HMS | Homosalate | 10.00 |
| | LexFilm Sun | Polyester-7 (and) | 5.00 |
| | | Neopentyl Glycol Diheptanoate | |
| | EMC 30 | Caprylic/Capric Triglyceride | 5.00 |
| | | (and) Castor Oil/PDI Copolymer | |
| B | Polyester Elastomer | | 47.00 |
| | Gel according to the | | |
| | invention | | |
| | Zetemol 512 | Isoamyl Laurate | 10.00 |
| | Cetiol C5C | Coco-Caprylate/Caprate | 5.00 |

### Example 7: Preparation of a Lipstick

Combined the ingredients in Phase A of Table 4 in a beaker and homogenized until pigments were fully grinded and dispersed. Added the rest of the ingredients in Phase B of Table 4 into the beaker except for the elastomer gel and heated to 85° C. Mixed until homogeneous. Once homogenous, slowly added the elastomer gel to the bulk and mixed until homogenous. Poured the bulk at 75° C.-80° C. into molds. See formulation specifics in Table 4.

TABLE 4

| | | | % |
|---|---|---|---|
| Phase | Ingredient | INCI Name | W/W |
| A | Florasun 90 | Helianthus Annuus | 18.00 |
| | | (Sunflower) Seed Oil | |
| | Castor Oil | Ricinus Communis | 15.00 |
| | | (Castor) Seed Oil | |
| | SunPURO ® Titanium | Titanium Dioxide | 8.40 |
| | Dioxide | | |
| | SunCROMA ® Red | Red Iron Oxide | 0.40 |
| | Iron Oxide | | |
| | SunPURO ® Yellow | Yellow Iron Oxide | 1.05 |
| | Iron Oxide | | |
| | SunPURO ® Black | Black Iron Oxide | 0.15 |
| | Iron Oxide | | |
| B | Harmonie Luxe 4 | Silica | 10.00 |
| | Powder | | |
| | Candelilla wax | Euphorbia Cerifera | 8.00 |
| | | (Candelilla) Wax | |
| | Sunflower Wax | Helianthus Annuus | 5.00 |
| | | (Sunflower) Seed Wax | |
| | Jojoba Wax | Hydrogenated Jojoba Oil | 4.00 |
| | FANCOL ® SHEA | Butyrospermum | 5.00 |
| | BUTTER | Parkii (Shea) Butter | |
| | Cetiol C5C | Coco-Caprylate/Caprate | 12.00 |
| | Polyester Elastomer | | 13.00 |
| | Gel according to the | | |
| | invention | | |

### Example 8: Preparation of a Foundation

Combined all the ingredients in Phase B of Table 5 into a beaker and homogenized until pigments were fully grinded and dispersed. Added the Bentone Luxe XO and Elastomer Gel to the mixture and homogenized until uniform. Added the Lexemul 515 MVB and heated to 75° C. and mixed until wax had fully melted. In a separate beaker, combined all of the ingredients in Phase A of Table 5 and heated to 75° C. while mixing until homogenous. Once both phases were at a temperature of 75° C., slowly added Phase A to Phase B under homogenization. Homogenized for an additional 2 minutes and let cool. See formulation specifics in Table 5.

TABLE 5

| | | | % |
|---|---|---|---|
| Phase | Ingredient | INCI Name | W/W |
| A | Water | Water | 32.00 |
| | Sodium Chloride | Sodium Chloride | 3.00 |
| | Spectrastat CGE Natural | Caprylhydroxamic | 2.00 |
| | MB | Acid (and) Caprylyl | |
| | | Glyceryl Ether (and) Propanediol | |
| | Glycerin | Glycerin | 5.00 |
| B | MCT Oil | Caprylic/Capric Triglyceride | 5.00 |
| | LexFeel Natural | Heptyl Undecylenate | 18.00 |
| | SunCROMA Titanium | Titanium Dioxide | 8.50 |
| | Dioxide | | |
| | SunCROMA Red Iron | Iron Oxide (CI 77491) | 0.15 |
| | Oxide | | |
| | SunCROMA Yellow Iron | Iron Oxide (CI 77492) | 1.30 |
| | Oxide | | |
| | SunCROMA Black Iron | Iron Oxide (CI 77499) | 0.05 |
| | Oxide | | |
| | Lexemul 515 MB | Glyceryl Stearate | 5.00 |
| | Bentone Luxe XO | C13-15 Alkane (and) | 10.00 |
| | | Disteardimonium | |
| | | Hectorite (and) Polyglyceryl-3 | |
| | | Polyricinoleate | |
| | Polyester Elastomer Gel | | 10.00 |

### Example 9: Preparation of a Moisturizing Stick

Combined all the raw materials in Table 7 in a beaker and heated to 85° C. Mixed until homogenous. Poured straight into packaging and let cool at room temperature to solidify. See formulation specifics in Table 6.

TABLE 6

| | | | % |
|---|---|---|---|
| Phase | Raw Material | INCI Name | W/W |
| | Vitamin E | Vitamin E | 1.00 |
| | FANCOR ® | Limnanthes Alba | 10.50 |
| | Meadowfoam | (Meadowfoam) Seed Oil | |
| | Seed Oil | | |
| | Jojoba Wax | Hydrogenated Jojoba Oil | 4.00 |
| | Sunflower Wax | Helianthus Annuus | 4.00 |
| | Polyester | (Sunflower) Seed Wax | 30.00 |
| | Elastomer Gel | | |
| | Candelilla Wax NF | Euphorbia Cerifera | 5.50 |
| | | (Candelilla) Wax | |
| | MCT | Caprylic/Capric Triglyceride | 40.00 |
| | FANCOL | Butyrospermum | 5.00 |
| | Shea butter | Parkii (Shea) Butter | |

### OTHER ASPECTS

All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

While the invention has been described in connection with specific aspects thereof, it will be understood that the invention is capable of further modifications and this application is intended to cover any variations, uses, or adaptations of the invention following, in general, the principles of the invention and including such departures from the present disclosure that come within known or customary practice within the art to which the invention pertains and can be applied to the essential features hereinbefore set forth, and follows in the scope of the claimed invention.

1. A polyester elastomer comprising a reaction product of:
(i) at least one di-carboxylic acid or tri-carboxylic acid;
(ii) at least one mono-carboxylic acid; and
(iii) at least one polyol.

2. The polyester elastomer of claim 1, wherein the at least one di-carboxylic acid is a compound of formula (IA) 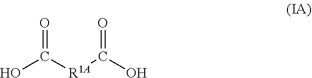
wherein
R^{1A} is C₂-C₅₂ alkyl group, C₂-C₅₂ heteroalkyl group, C₂-C₅₂ alkene group, C₂-C₅₂ heteroalkene group, C₃-C₅₂ cyclic group, or C₂-C₅₂ heterocyclic group.

3. The polyester elastomer of claim 2, wherein the di-carboxylic acid of formula (IA) is selected from the group consisting of succinic acid, glutaric acid, adipic acid, pimelic acid, azelaic acid, sebacic acid, brassylic acid, dodecanedioic acid, C₂₁ dimer acid, and C₃₆ dimer acid, maleic acid, fumaric acid, traumatic acid, and combinations thereof.

4. The polyester elastomer of claim 1, wherein the at least one tri-carboxylic acid is a compound of formula (IB) 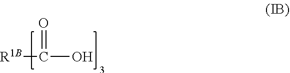
wherein
R^{1B} is C₂-C₅₂ alkyl group, C₂-C₅₂ heteroalkyl group, C₂-C₅₂ alkene group, C₂-C₅₂ heteroalkene group, C₃-C₅₂ cyclic group, or C₂-C₅₂ heterocyclic group.

5. The polyester elastomer of claim 4, wherein the tri-carboxylic acid of formula (IB) is selected from the group consisting of citric acid, C₅₄ trimer acid, and hydrogenated C₅₄ trimer acid.

6. The polyester elastomer of claim 1, wherein the at least one mono-carboxylic acid is a compound of formula (II) 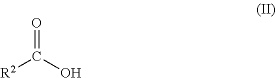
wherein
R² is C₂-C₅₂ alkyl group, C₂-C₅₂ heteroalkyl group, C₂-C₅₂ alkene group, C₂-C₅₂ heteroalkene group, C₃-C₅₂ cyclic group, or C₂-C₅₂ heterocyclic group.

7. The polyester elastomer of claim 6, wherein the mono-carboxylic acid of formula (II) is selected from the group consisting of caprylic acid, capric acid, lauric acid, myristic acid, palmitic acid, stearic acid, isostearic acid, behemic acid, oleic acid, linoleic acid, linolenic acid, and combinations thereof.

8. The polyester elastomer of claim 1, wherein the at least one polyol is a compound of formula (III) 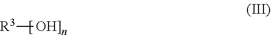
wherein
R³ is C₃-C₅₀ alkyl group, C₃-C₅₀ heteroalkyl group, C₃-C₅₀ alkene group, C₃-C₅₀ heteroalkene group, C₃-C₅₀ cyclic group, or C₃-C₅₀ heterocyclic group; and
n is an integer from 2 to 10.

9. The polyester elastomer of claim 8, wherein the polyol of formula (III) is selected from the group consisting of glycerol, diglycerol, polyglycerol, sorbitol, castor oil, hydrogenated castor oil, sugar alcohol, monosaccharide, disaccharides, oligosaccharide, polysaccharides, tannin, gallic acid, 1,2-propanediol, 1,3-propanediol, 1,4-butanediol, 1,2-pentanediol, 1,3-pentanediol, 1,4-pentanediol, 1,5-pentanediol, 1,2-hexanediol, 1,5-hexanediol, 1,6-hexanediol, C₃₆ dimer diol, hydrogenated C₃₆ dimer diol, and combinations thereof.

10. The polyester elastomer of claim 1, wherein the molar ratio of carboxylic acid functional groups (—COOH) from mono-carboxylic acid to hydroxyl functional groups (—OH) from polyol is from about 1:2 to about 1:16.

11. The polyester elastomer of claim 1, wherein the molar ratio of carboxylic acid functional groups (—COOH) from di-carboxylic acid or tri-carboxylic acid to hydroxyl functional groups (—OH) from polyol is from about 1.5:1 to about 1:4.

12. The polyester elastomer of claim 1, which has a gel fraction greater than 20%.

13. The polyester elastomer of claim 1, which has a swelling ratio of from about 1 gram/gram to about 15 gram/gram.

14. The polyester elastomer of claim 1, comprised of particles with a median diameter from about 1 μm to about 500 μm as measured by a laser diffraction particle size analyzer.

15. A method of preparing an elastomer comprising reacting:
(i) at least one di-carboxylic acid or tri-carboxylic acid;
(ii) at least one mono-carboxylic acid; and
(iii) at least one polyol;
to form a crosslinked polyester elastomer

16. The method of claim 15, wherein the reaction is carried out in the presence of at least one solvent or at least one emollient.

17. (canceled)

18. The method of claim 16, wherein the at least one solvent or the at least one emollient are selected from the group consisting of a triglyceride, a mono-ester, a di-ester, a citrate ester, an ether, a carbonate, a hydrocarbon, a silicone, and combinations thereof.

19. The method of claim 18, wherein the solvent or the emollient is:
(a) a triglyceride of formula (IV) 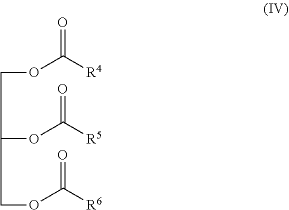
wherein
R⁴, R⁵, and R⁶ are independently C₁-C₃₅ alkyl group, C₁-C₃₅ heteroalkyl group, C₂-C₃₅ alkene group, or C₂-C₃₅ heteroalkene group; or
(b) a mono-ester of formula (V) 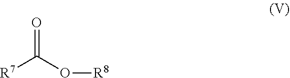
wherein
R⁷ and R⁸ are independently i-s-C₁-C₃₅ alkyl group, C₁-C₃₅ heteroalkyl group, C₂-C₃₅ alkene group, or C₂-C₃₅ heteroalkene group; or
(c) a di-ester of formula (VI) 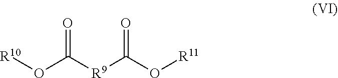
wherein
R⁹ is C₁-C₃₅ alkyl group, C₁-C₃₅ heteroalkyl group, C₂-C₃₅ alkene group, or C₂-C₃₅ heteroalkene group; and
R¹⁰ and R¹¹ are independently C₁-C₃₅ alkyl group, C₁-C₃₅ heteroalkyl group, C₂-C₃₅ alkene group, or C₂-C₃₅ heteroalkene group; or
(d) a di-ester of formula (VII) 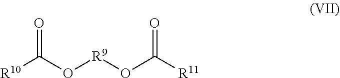
wherein
R⁹ is C₁-C₃₅ alkyl group, C₁-C₃₅ heteroalkyl group, C₂-C₃₅ alkene group, or C₂-C₃₅ heteroalkene group; and
R¹⁰ and R¹¹ are independently H, C₁-C₃₅ alkyl group, C₁-C₃₅ heteroalkyl group, C₂-C₃₅ alkene group, or C₂-C₃₅ heteroalkene group; or
(e) a di-ester of formula (VIII) 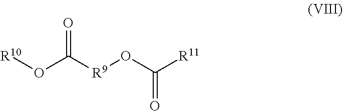
wherein
R⁹ and R¹⁰ are independently C₁-C₃₅ alkyl group, C₁-C₃₅ heteroalkyl group, C₂-C₃₅ alkene group, or C₂-C₃₅ heteroalkene group; and
R¹¹ is H, C₁-C₃₅ alkyl group, C₁-C₃₅ heteroalkyl group, C₂-C₃₅ alkene group, or C₂-C₃₅ heteroalkene group; or
(f) a citrate ester of formula (IX) 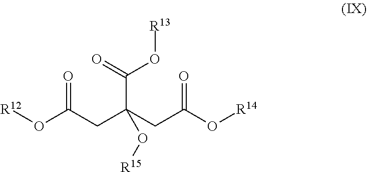
wherein
R¹², R¹³, R¹⁴, and R¹⁵ are independently H, C₁-C₃₅ alkyl group, C₁-C₃₅ heteroalkyl group, C₂-C₃₅ alkene group, or C₂-C₃₅ heteroalkene group,
wherein at least one of R¹², R¹³, R¹⁴, and R¹⁵ is not H; or
(g) an ether of formula (X) 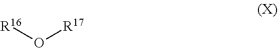
wherein
R¹⁶ and R¹⁷ are independently H, C₂-C₂₀ alkyl group, C₂-C₂₀ heteroalkyl group, C₂-C₂₀ alkene group, or C₂-C₂₀ heteroalkene group,
wherein at least one of R¹⁶ and R¹⁷ is not H; or
(h) a carbonate of formula (XI) 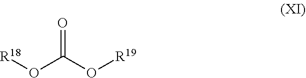
wherein
R¹⁸, and R¹⁹ are independently H, C₂-C₂₀ alkyl group, C₂-C₂₀ heteroalkyl group, C₂-C₂₀ alkene group, or C₂-C₂₀ heteroalkene group; or
(i) a hydrocarbon with a number of carbon atoms from C₄ to C₆₀.

20. The method of claim 18, wherein the solvent or the emollient is:
(a) a triglyceride selected from the group consisting of caprylic/capric triglyceride, triheptanoin, corn oil, soybean oil, olive oil, rape seed oil, cotton seed oil, coconut oil, almond oil, argon oil, rosehip oil, black seed oil, grape seed oil, avocado oil, apricot kernel oil, geranium oil, lavender oil, rosehip oil, macadamia oil, eucalyptus oil, sardine oil, herring oil, safflower oil, linseed oil, sunflower oil, olive oil, canola oil, sesame oil, cottonseed oil, palm oil, rapeseed oil, tung oil, fish oil, peanut oil, cuphea oil, milkweed oil, salicornia oil, whale oil, castor oil, and combinations thereof, or
(b) a mono-ester selected from the group consisting of coco-caprylate, coco-caprate, jojoba oil, jojoba esters, isopropyl jojobate, ethyl macadamiate, isoamyl laurate, heptyl undecylenate, methylheptyl isostearate, isostearyl isostearate, glyceryl ricinoleate, isostearyl palmitate, myristyl myristate, octyldodecyl myristate, octyldodecyl hydroxystearate, butyl myristate, ethylhexyl cocoate, ethylhexyl palmitate, ethylhexyl stearate, butyl stearate, decyl oleate, isocetyl behenate, isocetyl myristate, isocetyl palmitate, isocetyl stearate, isodecyl oleate, isopropyl isostearate, isopropyl myristate, isopropyl palmitate, oleyl oleate, propylene glycol laurate, octyldodecyl erucate, C₁₂-C₁₃ alkyl lactate, C₁₂-C₁₅ alkyl lactate, isostearyl lactate, glycereth-5-lactate, lauryl lactate, myristyl lactate, oleyl lactate, laureth-2-benzoate, C₁₂-C₁₅ alkyl benzoate, C₁₂-C₁₅ pareth-3-benzoate, dipropylene glycol benzoate, isodecyl salicylate, C₁₂-C₁₅ alkyl salicylate, tridecyl salicylate, ethylhexyl isononanoate, cetyl ethylhexanoate, isononyl isononanoate, isodecyl ethylhexanoate, isodecyl isononanoate, tridecyl ethylhexanoate, isotridecyl isononanoate, isostearyl isononanoate, cetearyl isononanoate, laureth-2-ethylhexanoate, cetearyl ethylhexanoate, isodecyl neopentanoate, isostearyl neopentanoate, nyristyl neopentanoate, isostearyl behenate, octyldodecyl neopentanoate, tridecyl neopentanoate, and combinations thereof; or
(c) a di-ester selected from the group consisting of diethyl succinate, dibutyl succinate, diethyhexyl succinate, diisopropyl sebacate, dimethyl sebacate, diethyl sebacate, dibutyl sebacate, diisostearyl dimer, diisostearyl malate, isostearyl stearoyl stearate, isocetyl stearoyl stearate, octyldodecyl stearoyl stearate, diethylhexyl malate, diethylhexyl maleate, dipropylene glycol dibenzoate, dicapryl adipate, dicaprylyl maleate, diisopropyl dimer, diisopropyl adipate, diisobutyl adipate, diisopropyl sebacate, diisostearyl dimer, diethyhexyl succinate, diethylene glycol diethylhexanoate, neopentyl glycol dicaprate, propylene glycol dicaprylate/dicaprate, neopentyl glycol diisostearate, neopentyl glycol diethylhexanoate, neopentyl glycol diheptanoate, and combinations thereof; or
(d) a citrate ester selected from the group consisting of tricaprylyl citrate, triisostearyl citrate, triisocetyl citrate, trioctyldodecyl citrate, triethyl citrate, tributyl citrate, acetyl triethyl citrate, acetyl tributyl citrate, trioctyldodecyl citrate, triisocetyl citrate, and combinations thereof; or
(e) an ether selected from the group consisting of dicaprylyl ether, didecyl ether, panthenyl ethyl ether, dicetyl ether, dimyristyl ether, distearyl ether, dilauryl ether, and combinations thereof; or
(f) a carbonate selected from the group consisting of dicaprylyl carbonate, diethyl hexyl carbonate, and combinations thereof; or
(g) a hydrocarbon selected from the group consisting of farnesene, hydrogenated farnesene, coconut alkanes, coconut/palm kernel alkanes, C₉-C₁₂ alkane, C₁₀-C₁₃ alkane, C₁₂-C₁₇ alkane, C₁₃-C₁₄ alkane, C₁₃-C₁₅ alkane, C₁₄-C₁₇ alkane, C₁₄-C₁₉ alkane, C₁₄-C₂₀ alkane, C₁₄-C₂₂ alkane, C₁₅-C₁₉ alkane, C₂₁-C₂₈ alkane, C₁₇-C₂₃ alkane, C₉-C₁₂ isoalkane, C₉-C₁₃ isoalkane, C₉-C₁₄ isoalkane, C₉-C₁₆ isoalkane, C₁₀-C₁₁ isoalkane, C₁₀-C₁₂ isoalkane, C₁₀-C₁₃ isoalkane, C₁₁-C₁₂ isoalkane, C₁₁-C₁₃ isoalkane, C₁₁-C₁₄ isoalkane, C₁₂-C₁₄ isoalkane, C₁₂-C₁₅ isoalkane, C₁₂-C₂₀ isoalkane, C₁₃-C₁₄ isoalkane, C₁₃-C₁₆ isoalkane, C₁₄-C₁₆ isoalkane, C₁₅-C₁₉ isoalkane, C₁₀-C₁₆ olefin, C₁₂-C₁₈ olefin, C₁₈-C₂₆ olefin, C₂₀ olefin, C₂₀-C₂₄ olefin, C₂₄-C₃₀ olefin, C₂₆-C₂₈ olefin, C₂₆-C₅₄ olefin, C₂₈-C₃₆ olefin, C₂₈-C₅₂ olefin, C₃₀-C₃₈ olefin, C₃₀-C₄₄ olefin, C₄-C₁₂ olefin, C₄-C₆ olefin, C₅-C₆ olefin, hydrogenated poly(C₆/C₁₀/C₁₄ olefin), hydrogenated poly(C₆-C₁₂ olefin), hydrogenated poly(C₆-C₁₄ olefin), hydrogenated poly(C₆-C₂₀ olefin), hydrogenated poly(C₈/C₁₂ olefin), poly(C₂₀-C₂₈ olefin), poly(C₃₀-C₄₅ olefin), poly(C₄-C₁₂ olefin), poly(C₆-C₁₄ olefin), hexadecene, C₃₂ alkane, C₃₂ isoalkane, C₅₄ alkane, C₅₄ isoalkane, diethylhexylcyclohexane, undecane, tridecane, tetradecane, pentadecane, hexadecane, octadecane, docosane, squalane, hydrogenated polyisobutene, polybutene, hydrogenated polydecene, hydrogenated didecene, mineral oil, liquidum, petrolatum, dodecane, isohexadecane, isododecane, isoeicosane, and combinations thereof.

21.-26. (canceled)

27. The method of claim 15, wherein the method is carried out in the presence of a catalyst selected from the group consisting of methanesulfonic acid, p-toluenesulfonic acid, benzene sulfonic acid, sulfuric acid, amidosulfonic acid, sulfamic acid, sodium bisulfate, phosphoric acid, hydrochloric acid, hydrobromic acid, nitric acid, sodium hydroxide, potassium hydroxide, sodium methoxide, sodium ethoxide, potassium methoxide, potassium ethoxide, bismuth neodecanoate, bismuth(III) citrate, bismuth(III) chloride, bismuth(III) acetate, bismuth(III) phosphate, tin chloride, tin-pyrone, dibutyltin dilaurate, di-n-butyl-oxo-stannane, butyl stannoic acid, zinc chloride, zinc bromide, zinc carboxylic salt, zinc oxide, zinc hydroxy nitrate salt, zinc hydroxy acetate, triethylamine, tripropylamine, cocamidopropyl dimethylamine, stearamidopropyl dimethylamine, isostearamidopropyl dimethylamine, Yb(OTf)₃, Sc(OTf)₃, Hf(OTf)₄, Bi(OTf)₃, Al(OTf)₃, Zn(OTf)₂, Mg(ClO₄)₂, Cu(OTf)₂, Ti(OCH(CH₃)₂)₄, and combinations thereof.

28. (canceled)

29. A composition comprising the polyester elastomer of claim 1 and one or more solvents or emollients.

30.-31. (canceled)

32. The composition of claim 29, which is in the form of a powder or a gel, wherein the gel has a storage modulus (G′) from about 10 Pa to about 100,000 Pa, and/or a loss modulus (G″) from about 10 Pa to about 100,000 Pa each as measured by rheometer within linear viscoelastic region using dynamic rheology.

33. (canceled)

34. A personal care formulation comprising the composition of claim 29, which is selected from the group consisting of a deodorant, an antiperspirant, a skin cream, a facial cream, a hair shampoo, a hair conditioner, a mousse, a hair styling gel, a hair spray, a protective cream, a lipstick, a facial foundation, blushes, makeup, a mascara, a skin care lotion, a moisturizer, a facial treatment, a personal cleanser, a facial cleanser, a bath oil, a perfume, a shaving cream, a pre-shave lotion, an after-shave lotion, a cologne, a sachet, and a sunscreen.

35. (canceled)

36. The method of claim 15, wherein the method further comprises:
(iv) combining the crosslinked polyester elastomer with at least one solvent or emollient thereby forming a swollen crosslinked polyester elastomer; and
(v) subjecting the swollen crosslinked polyester elastomer to shear force thereby forming a polyester elastomer gel.

37. (canceled)
